(19) United States
(12) Patent Application Publication
(10) Pub. No.: US 2026/0137741 A1
(43) Pub. Date: May 21, 2026

(54) MICROBIAL OILS HAVING HIGH EPA CONTENT AND METHODS OF MAKING THE SAME

(71) Applicant: DSM IP Assets B.V., Maastricht (NL)

(72) Inventors: Daniel Xiao Dong, Ellicott City, MD (US); Wouter Adrianus Van Winden, Den Haag (NL); Adam Burja, Halifax (CA); Jose R. Garcia, Lorton, VA (US); Michael Anthony Johnson, Columbia, MD (US)

(73) Assignee: DSM IP Assets B.V., Maastricht (NL)

(21) Appl. No.: 19/446,790

(22) Filed: Jan. 12, 2026

Related U.S. Application Data
(63) Continuation of application No. PCT/US2024/038047, filed on Jul. 15, 2024.
(60) Provisional application No. 63/513,763, filed on Jul. 14, 2023.
(60) Provisional application No. 63/545,469, filed on Oct. 24, 2023.
(60) Provisional application No. 63/610,789, filed on Dec. 15, 2023.

(30) Foreign Application Priority Data
Dec. 19, 2023 (EP) 23218376.4

(51) Int. Cl. A61K 36/02

(52) U.S. Cl. CPC A61K 36/02

(57) ABSTRACT

The present disclosure describes microbial oil compositions that can include a biomass present in a culture medium. The biomass can include a microbial oil enriched in EPA. The present disclosure also describes methods of producing microbial oils enriched in EPA. The present disclosure also describes nutritional oil compositions including the microbial oils and nutritional oil concentrates prepared from the nutritional oil compositions or microbial oils.

### CROSS-REFERENCE TO RELATED APPLICATIONS

This application is a continuation of PCT Application No. PCT/US2024/038047, filed 15 Jul. 2024, which claims the benefit of U.S. Provisional Patent Application Nos. 63/610,789, filed on 15 Dec. 2023, 63/545,469, filed on 24 Oct. 2023, and 63/513,763, filed on 14 Jul. 2023, and the benefit of European Application No. 23218376, filed 19 Dec. 2023 each of which is incorporated herein by reference in its entirety.

### TECHNICAL FIELD

The present disclosure relates to microbial oils and nutritional oil compositions enriched in EPA. The present disclosure also relates to methods of making microbial oils and nutritional oil compositions enriched in EPA.

### BACKGROUND

Omega-3 and omega-6 fatty acids are the two main classes of polyunsaturated fatty acids (PUFAs). PUFAs are designated by their number of carbon atoms and double bonds. For example, eicosapentaenoic acid is known as C20:5n3 because it has 20 carbon atoms and 5 carbon-carbon double bonds, where the carbon-carbon double bond most proximate to the methyl end of the fatty acid chain is positioned at the third carbon from the methyl end. Alpha linoleic acid (ALA, C18:3n-3), eicosapentaenoic acid (EPA, 20:5n-3), and docosahexaenoic acid (DHA, 22:6n-3) are some of the most well-known omega-3, or n-3, fatty acids. Omega-6 fatty acids, or n-6 fatty acids, are PUFAs where the carbon-carbon double bond most proximate the methyl end of the fatty acid chain is positioned at the sixth carbon from the methyl end. Linoleic acid (LA, 18:2n-6) and arachidonic acid (ARA, 20:4n-6) are two of the most well-known omega-6 fatty acids.

The omega-3 fatty acids eicosapentaenoic acid (EPA, 20:5n-3) and docosahexaenoic (DHA, 22:6n-3) have long been recognized as having high nutritional importance. Historical data collected on populations consuming relatively high amounts of these PUFAs suggests that these groups experienced lower rates of chronic disease, including cardiovascular disease, arthritis, asthma, diabetes, and others.

In particular, EPA performs a variety of important functions in biological membranes and is a precursor of various lipid regulators in cellular metabolism. For example, EPA plays an important role as a precursor of a group of eicosanoids that facilitates regulating developmental and regulatory physiology. Additionally, EPA can help prevent atherosclerosis by decreasing the level of low-density lipoproteins in the circulatory system. EPA also appears to reduce the likelihood of heart attack and arrhythmias due to its influence on the electrical behavior and chemical responses of the heart. EPA has also been shown to be useful in the treatment of mental disorders such as schizophrenia and bipolar disorder.

Unfortunately, the human body is unable to form carbon-carbon double bonds more proximate than the ninth carbon from the methyl end of a fatty acid chain. Therefore, ALA and LA are both considered essential fatty acids because they can be converted into other omega-3 and omega-6 fatty acids in the body. However, the conversion of ALA to EPA and DHA in the body is very limited, making it very important to obtain these omega-3 fatty acids from the diet.

Fish oil has long been the richest source of EPA for consumers, but EPA and DHA are originally synthesized by microalgae, not by the fish. When fish consume phytoplankton that previously consumed PUFA-rich microalgae, the fish accumulate omega-3 fatty acids in their tissues. However, the quality of fish oil depends on the fish species, the season, and the geographical locations of the catching sites. Fish stocks are also subject to seasonal and climatic variation, as well as chronic over-fishing. Moreover, fish oils have a peculiar taste and odor and are primarily obtained from fish livers, which is the same organ in which deleterious synthetic compounds ingested by the fish are concentrated. As such, fish oil is expensive to recover from fish and subsequently purify for human consumption. Therefore, alternative, commercially relevant sources of EPA are needed.

Thraustochytrids are microorganisms of the order Thraustochytriales. Thraustochytrids include members of the genus Schizochytrium and Thraustochytrium and have been recognized as an alternative source of omega-3 fatty acids, including DHA and EPA. Oils produced from these marine heterotrophic microorganisms often have simpler PUFA profiles than corresponding fish oils. Strains of Thraustochytrids have been reported to produce omega-3 fatty acids as a high percentage of the total fatty acids produced by the organisms. However, isolated Thraustochytrids vary in the identity and amounts of long chain polyunsaturated fatty acids (LC-PUFAs) produced, such that some strains can have undesirable levels of omega-6 fatty acids and/or can demonstrate low productivity in culture. Additionally, many Thraustochytrids preferentially produce DHA and produce only minimal amounts of EPA. As such, a continuing need exists for methods of producing commercially-relevant amounts of EPA from microbial sources, such as Thraustochytrids, for example.

### BRIEF SUMMARY

The present disclosure describes microbial oil compositions that can include a biomass present in a culture medium optionally at a concentration of at least 20 g/L, or from 20 g/L to 200 g/L. The biomass can include a microbial oil optionally present at a concentration of at least 45 wt. %, or from 45 wt % to 80 wt %, based on a total dry cell weight of the biomass.

The present disclosure also describes methods of producing microbial oils. The method can include cultivating a microorganism in a culture medium to form a biomass; inducing the microorganism to produce a lipid component enriched in EPA; extracting the lipid component from the biomass to obtain the microbial oil; and optionally, refining the microbial oil to reduce an amount of a free fatty acid, an impurity, a saturated fat or fatty acid, a colorant, or a combination thereof in the microbial oil.

The present disclosure also describes nutritional oil compositions including the microbial oils described herein.

In some examples, the microbial oil can include EPA in an amount of at least 200 mg/g or at least 300 mg/g based on the total weight of the microbial oil.

In some examples, the microbial oil can include EPA and DHA at a weight ratio of greater than or equal to 1.0 EPA/DHA or greater than or equal to 1.2 EPA/DHA.

In some examples, the microbial oil can include DPAn-3 at a weight ratio with DHA of at least 1.3 DHA/DPAn-3.

In some examples, the microbial oil can include EPA and DHA in a combined amount of greater than or equal to 300 mg/g based on the total weight of the microbial oil.

In some examples, the microbial oil can include DHA in an amount greater than or equal to 125 mg/g based on the total weight of the microbial oil.

In some examples, the microbial oil can include 70 wt. % or greater of triacylglycerols (triglycerides) based on the total weight of the microbial oil.

In some examples, the microbial oil can include DPAn-6.

In some examples, the microbial oil can include DPAn-3 and DPAn-6 at a weight ratio of at least 1.5 DPAn-3/DPAn-6.

In some examples, the microbial oil can include omega-3 PUFAs in an amount greater than or equal to 500 mg/g based on the total weight of the microbial oil.

In some examples, the microbial oil can include palmitic acid in an amount greater than or equal to 30 mg/g based on the total weight of the microbial oil.

In some examples, the microbial oil can include stigmasterol.

In some examples, the microbial oil can be combined with an antioxidant.

In some examples, the microbial oil can be combined with a nutritional or pharmaceutical carrier.

Each of the foregoing examples is considered to be non-limiting. Furthermore, each of the foregoing examples can be combined with any of the other examples unless the context clearly indicates otherwise.

### DETAILED DESCRIPTION

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an,” and “the” include express support for plural referents unless the context clearly dictates otherwise. Thus, for example, reference to “a fatty acid” or “the fatty acid” can include a plurality of such fatty acids.

Unless otherwise specified, all numerical parameters are to be understood as being prefaced and modified in all instances by the term “about,” in which the numerical parameters possess the inherent variability characteristic of the underlying measurement techniques used to determine the numerical value of the parameter. In some non-limiting examples, the inherent variability of the underlying measurement technique can be plus or minus 1%, 5%, 10%, or more (relative percent), depending on the particular measurement technique employed.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to 5” should be interpreted to include not only the explicitly recited values of 1 to 5, but also include individual values and sub-ranges within the indicated range. Thus, included in this numerical range are individual values such as 1, 2, 3, 4, and 5 and sub-ranges such as from 1 to 3, from 2 to 4, from 3 to 5, etc. This same principle applies to ranges reciting only one numerical value as a minimum or a maximum. Furthermore, such an interpretation should apply regardless of the breadth of the range or the characteristics being described.

Reference throughout this written description to “an example” means that a particular feature, component, or characteristic described in connection with the example is included in at least one embodiment. Thus, appearances of the phrases “in an example” or “in one example” in various places throughout this written description are not necessarily all referring to the same embodiment.

### Microorganisms

The methods described herein can be performed, and the microbial oils or nutritional oil compositions described herein can be obtained, via a suitable biomanufacturing process employing one or more suitable microorganisms. For example, microbial oils can be produced and obtained from algae, microalgae, fungi (including yeast), bacteria, and/or protists.

In some specific examples, the microbial oils described herein can be produced, or largely produced, by a microalgae. In some additional specific examples, the microbial oils described herein can be produced, or largely produced, by a Thraustochytrid, which, at the time of this disclosure, is classified as a type of microalgae.

For purposes of the present disclosure, strains described as Thraustochytrids include organisms of the taxonomic order Thraustochytrialies. In some further examples, Thraustochytrids can include organisms of the taxonomic family Thraustochytriidae (or Thraustochytriaceae). In still further examples, Thraustochytrids can include organisms of the taxonomic genus Thraustochytrium, Ulkenia, Schizochytrium, Japonochytrium, Aplanochytrium, Althornia, Elina, Aurantiochytrium, Oblongichytrium, Botryochytrium, Parietichytrium, Sicyoidochytrium, or a combination thereof.

In some further examples, the microbial oils described herein can be produced, or at least largely produced, from a microorganism of a species, or a microorganism derived from a species, of the genus Thraustochytrium, Ulkenia, Schizochytrium, Japonochytrium, and/or Aplanochytrium.

In some specific examples, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a single microorganism source. In some examples, the single microorganism source can be a heterotrophic microorganism. In some examples, the single microorganism source can include a species from the genus Thraustochytrium, Ulkenia, Schizochytrium, Japonochytrium, Aplanochytrium, Althornia, Elina, Aurantiochytrium, Oblongichytrium, Botryochytrium, Parietichytrium, or Sicyoidochytrium. In some further examples, the single microorganism source can include a species of the genus Thraustochytrium, Ulkenia, Schizochytrium, Japonochytrium, and/or Aplanochytrium. In still further examples, the single microorganism source can include a species from the genus Thraustochytrium. In still further examples, the single microorganism source can include a species from the genus Schizochytrium.

In some specific examples, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a microorganism of a species, or a microorganism derived from a species, of the genus Thraustochytrium. Non-limiting examples of species from the genus Thraustochytrium can include Thraustochytrium arudimentale, Thraustochytrium aureum, Thraustochytrium benthicola, Thraustochytrium globosum, Thraustochytrium kinnei, Thraustochytrium motivum, Thraustochytrium multirudimentale, Thraustochytrium pachydermum, Thraustochytrium proliferum, Thraustochytrium roseum, Thraustochytrium striatum, and Thraustochytrium sp.

In some specific examples, at least 50 wt. %, at least 60 wt. %, at least 70 wt. %, at least 80 wt. %, at least 90 wt. %, or at least 95 wt. % of the lipid content (e.g., the microbial oil) of the microbial oil compositions or the nutritional oil compositions can be produced from a microorganism of a species, or a microorganism derived from a species, of the genus Ulkenia. Non-limiting examples of species from the genus Ulkenia can include Ulkenia amoeboidea, Ulkenia kerguelensis, Ulkenia minuta, Ulkenia profunda, Ulkenia radiata, Ulkenia sailens, Ulkenia sarkariana, Ulkenia schizochytrops, Ulkenia visurgensis, Ulkenia yorkensis, and Ulkenia sp.

In some specific examples, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a microorganism of a species, or a microorganism derived from a species, of the genus Schizochytrium. Non-limiting examples of species from the genus Schizochytrium can include Schizochytrium aggregatum, Schizochytrium limnaceum, Schizochytrium mangrovei, Schizochytrium minuturn, Schizochytrium octosporum, Schizochytrium sp.

In some specific examples, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a microorganism of a species, or a microorganism derived from a species, of the genus Japonochytrium. Non-limiting examples of species from the genus Japonochytrium can include Japonochytrium marinum, and Japonochytrium sp.

In some specific examples, at least 50 wt. %, at least 60 wt. %, at least 70 wt. %, at least 80 wt. %, at least 90 wt. %, or at least 95 wt. % of the lipid content (e.g., the microbial oil or nutritional oil) the microbial oil compositions or the nutritional oil compositions can be produced from a microorganism of a species, or a microorganism derived from a species, of the genus Aplanochytrium. Non-limiting examples of species from the genus Aplanochytrium can include Aplanochytrium haliotidis, Aplanochytrium kerguelensis, Aplanochytrium profunda, Aplanochytrium stocchinoi, and Aplanochytrium sp.

In another specific example, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a Thraustochytrid of a species, or a Thraustochytrid derived from a species, deposited under ATCC Accession No. PTA-6245, PTA-9695, PTA-10208, PTA-10209, PTA-10210, PTA-10211, PTA-10212, PTA-10213, PTA-10214, PTA-10215, or a combination thereof.

In some examples, at least 50 wt. %, at least 60 wt. %, at least 70 wt. %, at least 80 wt. %, at least 90 wt. %, or at least 95 wt. % of the lipid content (e.g., the microbial oil or nutritional oil) of the microbial oil compositions or the nutritional oil compositions can be produced from a genetically modified Thraustochytrid, such as, for example, Thraustochytrid genetically modified to enhance PUFA production. Non-limiting examples of such microorganisms are described in U.S. Pat. Nos. 8,309,796; 8,426,686; 8,859,855; 8,940,884; 9,012,616; 9,382,521; 9,133,463; 9,540,666; 9,873,880; 10,085,465; 10,087430; and 10,973,837, each of which is incorporated herein by reference.

In some examples, the lipid content obtained from a microorganism can be mixed or blended with a nutritional oil obtained from a different strain, a different species (e.g., Nannochloropsis spp., Yarrowia spp., Thraustochytrium spp., Ulkenia spp., Schizochytrium spp., Japonochytrium spp., Aplanochytrium spp., etc.), a plant source, a marine source (e.g., fish, krill, etc.), an animal source, or other suitable source to obtain a nutritional oil composition or microbial oil composition as described herein.

### Culture and Fermentation

The microorganisms described herein, such as Thraustochytrids, for example, can be cultured in large scale industrial bioreactors. The microorganisms can be cultured under conditions that increase biomass and/or production of a compound of interest (e.g., total lipid content, total EPA content, etc.). For example, the production of desirable lipids can be enhanced by culturing cells according to methods that involve a shift of one or more culture conditions in order to obtain higher quantities of desirable polyunsaturated fatty acids (PUFAs), such as EPA.

In further detail, PUFAs can typically be produced by culturing the microorganisms in a two-phase process that includes a growth phase, in which cell biomass is increased, followed by a production phase, in which the biomass synthesizes lipids. In some examples, the transition between the growth phase and the production phase can be defined as the point at which the microorganism is starved of a supply of a nutrient other than carbon, such as when nitrogen is exhausted from the culture medium, for example. The lipid component can be considered a microbial oil and can be separated from the biomass for use as a nutritional oil or for use as a component of a nutritional oil composition.

Typically, the growth phase is performed under high-oxygen conditions, while the production phase is performed under low-oxygen conditions. A variety of other micro- and macro-nutrients can be included in the culture medium and optionally adjusted between the growth phase and the production phase to optimize biomass growth during the growth phase and lipid production during the production phase. For example, in some cases, it may be desirable to adjust oxygen concentration, carbon to nitrogen ratio, temperature, and/or other factors between the growth phase and the production phase to increase lipid production and/or production of a particular fatty acid, for example, during the production phase. In some specific examples, the growth phase can include higher oxygen, lower carbon to nitrogen ratio, and higher temperature relative to the production phase.

More generally, the microorganisms can be cultured in saline media. In some examples, the microorganisms can be cultured in media having a salt concentration of from 2 g/L to 50 g/L. In some additional examples, the microorganisms can be cultured in media having a salt concentration of from 2 g/L to 35 g/L or from 18 g/L to 50 g/L. In still additional examples, the microorganisms can be cultured in media having a salt concentration from 5 g/L to 15 g/L or 20 g/L. In some examples, the culture media can include NaCl, natural sea salt, artificial sea salt, artificial sea water, the like, or a combination thereof.

The chloride concentration in the culture media can generally be maintained at relatively low levels. For example, in some cases, the culture media can include non-chloride-containing sodium salts (e.g., sodium sulfate, for example) as a source of sodium. In some examples, at least a portion of the sodium content is supplied by a non-chloride salt. In some specific examples, at least 25 wt. %, at least 50 wt. %, or at least 75 wt. % of the sodium content is supplied by a non-chloride salt. Non-limiting examples of non-chloride salts can include soda ash (mixture of sodium carbonate and sodium oxide), sodium carbonate, sodium bicarbonate, sodium sulfate, the like, or a combination thereof. In some additional specific examples, the chloride concentration in the culture medium can be less than 3 g/L, less than 500 mg/L, less than 250 mg/L, or less than 120 mg/L. Additional details can also be found in U.S. Pat. Nos. 5,340,742 and 6,607,900, each of which is incorporated herein by reference.

The culture media can also include a variety of carbon sources. Non-limiting examples of carbon sources can include a fatty acid, a lipid, glycerol, an acylglycerol, glucose, starch, cellulose, hemicellulose, fructose, dextrose, xylose, lactulose, galactose, maltotriose, maltose, lactose, glycogen, gelatin, acetate, m-inositol, galacturonic acid, L-fucose, gentiobiose, glucosamine, alpha-D-glucose-1-phosphate, cellobiose, dextrin, alpha-cyclodextrin, sucrose, maltitol, erythritol, adonitol, N-acetyl-D-galactosamine, N-acetyl-D-glucosamine, N-acetyl-beta-D-mannosamine, biomass, a carbon-containing waste stream, the like, or a combination thereof. In some specific examples, the carbon source can be or include glucose. In some specific examples, the carbon source can be or include dextrose. In some specific examples, the carbon source can be or include sucrose. In some specific examples, the carbon source can be or include glycerol.

Typically, the culture media can include a carbon source at a concentration of 5 g/L to 200 g/L. In some examples, the culture media can include a carbon source at a concentration of 5 g/L to 100 g/L, from 10 g/L to 50 g/L, or from 15 g/L to 30 g/L.

A variety of nitrogen sources can be used with the culture media. Non-limiting examples of nitrogen sources can include ammonium solutions (e.g., NH4 in H2O), ammonium or amine salts (e.g., NH2SO4, (NH4)3PO4, NH4NO3, NH4OCH2CH3), peptone, tryptone, yeast extract, malt extract, fish meal, sodium glutamate, soy extract, casamino acids, distiller grains, the like, or a combination thereof.

In some specific examples, nitrogen can be included in the culture media at a concentration of from 5 g/L to 30 g/L at the beginning of the first phase (growth phase). In some additional examples, the nitrogen concentration can be lower at the beginning of the production phase than the nitrogen concentration at the beginning of the growth phase.

Typically, the culture media can have a pH of from 6.5 to 9.5. In some additional examples, the culture media can have a pH of from 6.5 to 8.0, from 6.8 to 7.8, or from 7.0 to 8.5. In some examples, the pH of the culture media can be adjusted up or down between the growth phase and the production phase to facilitate greater biomass growth in the growth phase and/or greater lipid production during the production phase. In some specific examples, the pH can be shifted up from a range from 6.5 to 7.5 in the growth phase to a higher pH range from 7.5 to 8.5 during the production phase.

Typically, the microorganisms can be cultured in the culture media at a temperature from 15° C. to 30° C. In some further examples, the microorganisms can be cultured in the culture media at a temperature of from 18° C. to 28° C., from 20° C. to 25° C., from 18° C. to 22° C., or from 21° C. to 23° C. In some examples, the temperature of the culture media can be adjusted up or down between the growth phase and the production phase to facilitate greater biomass growth in the growth phase and/or greater lipid production during the production phase.

A variety of fermentation outcomes can be adjusted by altering macronutrients and/or the timing of their addition. For example, altering macronutrients and/or the timing of their addition can, in some cases, increase a concentration and/or titer of lipid and/or a specific PUFA of interest (e.g., EPA).

In some specific examples, lowering the initial sodium to potassium (Na:K) weight ratio (e.g., by lowering Na mass or increasing K mass) in the culture media as compared to a traditional initial Na:K weight ratio can increase a concentration of EPA in the lipid produced by the microorganism. In some specific examples, the initial Na:K weight ratio in the culture media can be from 1:1 to 8:1. In some additional examples, the initial Na:K weight ratio in the culture media can be from 1.5:1 to 6:1 or from 1.75:1 to 4:1 or from 2:1 to 3:1. The initial Na:K weight ratio can be calculated based on the amounts of Na and K present in the various constituents included in the culture medium at the beginning of the growth phase. It is noted that the final Na:K weight ratio may be somewhat altered as compared to the initial Na:K weight ratio if a Na- and/or a K-bearing additive is used as a pH adjuster during the fermentation run (e.g., to maintain a particular pH during fermentation (or a phase of fermentation) or to adjust the pH for the production phase, for example).

In some additional specific examples, lowering the total nitrogen to phosphorus (N:P) weight ratio (e.g., by decreasing the N mass or increasing the P mass) in the culture media as compared to a traditional total N:P weight ratio can increase total lipid content produced by the microorganism and/or the concentration of EPA in the lipid produced by the microorganism. By “total N:P weight ratio,” it is meant the total amount, by weight, of nitrogen added to the culture medium over the course of the fermentation run relative to the total amount, by weight, of phosphorus added to the culture medium over the course of the fermentation run. The respective weights of N and P can be calculated based on the amounts of N and P present in the various constituents added to the culture media over the course of the fermentation run. In some examples, the total N:P weight ratio can be from 7:1 to 15:1. In still other examples, the total N:P weight ratio can be from 7:1 to 9:1, from 8:1 to 10:1, from 9:1 to 11:1, from 10:1 to 12:1, or from 11:1 to 13:1.

In some additional examples, the EPA concentration of the lipid produced by the microorganism can be increased by supplementing the culture media with CO2. Generally, CO2 can be introduced to the culture media to achieve a peak amount of dissolved CO2 in the culture media of greater than 50 ppm, greater than 100 ppm, greater than 150 ppm, greater than 200 ppm, or greater than 250 ppm based on the total mass of the culture media. In some examples, CO2 can be introduced to the culture media to achieve a peak amount of dissolved CO2 in the culture media of from 50 ppm to 300 ppm based on the total mass of the culture media. In still additional examples, CO2 can be introduced to the culture media to achieve a peak amount of dissolved CO2 in the culture media of from 50 ppm to 200 ppm, from 100 ppm to 250 ppm, or from 150 ppm to 300 ppm based on the total mass of the culture media. In some examples, CO2 can be introduced to the culture media to achieve the peak amount of CO2 during the growth phase. In some specific examples, CO2 can be introduced to the culture media to achieve the peak amount of CO2 during the last half of the growth phase. In some examples, CO2 can be introduced to the culture media to achieve the peak amount of CO2 during the production phase. In some specific examples, CO2 can be introduced to the culture media to achieve the peak amount of CO2 during the first half of the production phase.

Additionally, in some examples, CO2 can be added to the culture media in an amount to achieve an average dissolved CO2 concentration over the entire fermentation run of at least 100 ppm, at least 120 ppm, at least 140 ppm, at least 160 ppm, or at least 180 ppm. In some additional examples, CO2 can be added to the culture media in an amount to achieve an average dissolved CO2 concentration over the entire fermentation run of from 100 ppm to 220 ppm, from 120 ppm to 200 ppm, or from 140 ppm to 180 ppm. In some cases, elevated dissolved CO2 concentrations can adversely affect cell growth during the growth phase. As such, in some examples, the average dissolved CO2 concentration during the production phase can be greater than the average dissolved CO2 concentration during the growth phase. As is well understood in the art, the amount of dissolved CO2 in the culture media can be calculated using Henry's Law based on the amount of CO2 in the outlet gas from the fermenter.

The microorganisms can typically be cultivated to achieve a biomass concentration of at least 20 g/L by the end of the growth phase. In some further examples, the microorganisms can be cultivated to achieve a biomass concentration of at least 40 g/L, at least 60 g/L, at least 80 g/L, at least 90 g/L, or at least 100 g/L by the end of the growth phase. In some further examples, the microorganisms can be cultivated to achieve a biomass concentration of from 20 g/L to 200 g/L, from 40 g/L to 180 g/L, from 60 g/L to 160 g/L, or from 80 g/L to 140 g/L by the end of the growth phase. Biomass concentration can be determined based on the mass of a washed (e.g., to remove residual salts, carbohydrates, etc.) and lyophilized biomass obtained per unit volume of fermentation media.

The microorganisms can typically be cultivated to achieve a lipid content (or fat content or oil content) of at least 20 wt. % based on a total dry cell weight of the microorganism biomass by the end of the production phase. In some further examples, the microorganisms can be cultivated to achieve a lipid content (or fat content or oil content) of at least 30 wt. %, at least 40 wt. %, at least 50 wt. %, at least 55 wt. %, at least 60 wt. %, or at least 65 wt. % based on the total dry cell weight of the biomass by the end of the production phase. In additional examples, the microorganisms can be cultivated to achieve a lipid content (or fat content or oil content) of from 20 wt. % to 80 wt. %, from 30 wt. % to 70 wt. %, from 40 wt. % to 60 wt. %, from 35 wt. % to 55 wt. %, or from 45 wt. % to 65 wt. % based on the total weight of the biomass by the end of the production phase. Dry cell weight is based on the mass of a washed (e.g., to remove residual salts, carbohydrates, etc.) and lyophilized biomass.

In some examples, the volume of the culture media can be at least 2 liters, at least 10 liters, at least 50 liters, at least 100 liters, at least 200 liters, at least 200 liters, at least 1000 liters, at least 10,000 liters, at least 20,000 liters, at least 50,000 liters, at least 100,000 liters, at least 150,000 liters, at least 200,000 liters, or at least 250,000 liters. In some examples, the culture media is from 2 liters to 300,000 liters, from 10 liters to 200,000 liters or 250,000 liters, from 50 liters to 100,000 liters or 150,000 liters, from 100 liters to 50,000 liters, from 500 liters to 20,000 liters, or from 1000 liters to 10,000 liters.

The microorganism cells can be cultivated for a cultivation period anywhere from 1 day to 60 days. In some examples, the microorganism cells can be cultivated for a cultivation period of from 1 day to 14 days, from 2 days to 13 days, from 3 days to 12 days, from 4 days to 11 days, from 5 days to 10 days, from 6 days to 9 days, or from 7 days to 8 days.

In some examples, cultivation can include aeration-shaking culture, shaking culture, stationary culture, batch culture, semi-continuous culture, continuous culture, rolling batch culture, wave culture, or the like. In some additional examples, cultivation can be performed used a conventional agitation-fermenter, a bubble column fermenter (batch or continuous), a wave fermenter, or the like.

Cultures can be aerated using a variety of methods, such as general agitation, shaking, mixing, bubbling, etc. Dissolved O2 can typically be higher during the growth phase than the production phase. In some examples, decreasing the dissolved 02 content in the culture media during the production phase can increase the concentration of the EPA in the lipid produced by the microorganism.

Additional methods and details related to culture and fermentation can be found in U.S. Pat. Nos. 8,669,090; 9,045,785; 9,453,172; and 10,435,725 and U.S. Patent Publication No. 2021/0171991, each of which is incorporated by reference in their entirety.

### Pasteurization

Optionally, the biomass resulting from the formation process can be pasteurized to kill the cells and inactivate undesirable substances present in the biomass. For example, the biomass can be pasteurized to inactivate substances that can lead to the degradation of desirable lipids or other compounds. The biomass can be pasteurized in the fermentation media or isolated from the fermentation media for pasteurization. Pasteurization can be performed by heating the biomass and/or fermentation media to temperature sufficient to kill the cells and/or inactivate certain undesirable substances. In some examples, pasteurization can be performed at a temperature of from 50° C. to 90° C. for a pasteurization period. In other examples, pasteurization can be performed at a temperature of from 50° C. to 70° C., from 60° C. to 80° C., or from 70° C. to 90° C. for a pasteurization period.

The pasteurization period can typically be less than or equal to 120 minutes. In some examples, the pasteurization period can be from 30 minutes to 120 minutes. In some specific examples, the pasteurization period can be from 30 minutes to 90 minutes, from 45 minutes to 105 minutes, or from 60 minutes to 120 minutes. In still additional examples, the pasteurization period can be from 45 minutes to 75 minutes, or from 55 minutes to 90 minutes.

Pasteurization can be performed using a variety of suitable methods, including indirect and/or direct heating methods. In some examples pasteurization can be performed using an indirect heating method, such as via a heat transfer jacket or plate, for example. In some examples, pasteurization can be performed using a direct heating method, such as direct steam injection, for example.

### Harvesting

Optionally, the biomass can be harvested according to a variety of conventional methods. As non-limiting examples, the biomass can be collected using centrifugation (e.g., solid-ejecting centrifugation), filtration (e.g., cross-flow filtration), and/or other suitable harvesting methods. In some examples, harvesting can also employ the use of a precipitation agent (e.g., sodium phosphate or calcium chloride) to accelerate collection of the biomass.

In some examples, the biomass can also be washed with water. In some examples, the biomass can be concentrated to achieve a desired solids content. Additional methods and details related to harvesting biomass are also discussed in U.S. Pat. No. 5,130,242 and U.S. Patent Publication No. 2002/0001833, each of which is incorporated herein by reference.

### Hydrolysis

Cell hydrolysis (i.e., biomass cell disruption) can be performed using a variety of methods. In some examples, cell hydrolysis can be performed using chemical, enzymatic, and/or mechanical methods.

Chemical hydrolysis can be performed in a variety of ways, such as via acid hydrolysis, base hydrolysis, detergent hydrolysis, or the like. In some examples, chemical hydrolysis can be performed by acid hydrolysis by adding an acid to the biomass mixture. Typically, acid hydrolysis can include washing the biomass with water. Acid can be added to the washed biomass mixture. In some examples, the biomass is not dried prior to adding the acid. In other examples, the biomass is dried prior to adding the acid. Non-limiting examples of acids that can be used in acid hydrolysis can include sulfuric acid, hydrochloric acid, phosphoric acid, hydrobromic acid, nitric acid, perchloric acid, or other strong acids.

The acid can be added to the biomass mixture in a sufficient amount and for a sufficient incubation period to hydrolyze the cells. In some examples, the acid can be added to the biomass in an amount to achieve a final concentration of acid of from 100 mM to 200 mM. In some additional examples, the acid can be added to the biomass in an amount to achieve a final concentration of acid of from 100 mM to 140 mM, from 120 mM to 160 mM, from 140 mM to 180 mM, or from 160 mM to 200 mM.

The incubation period to hydrolyze the cells is further impacted by the temperature at which acid hydrolysis is conducted. In some examples, acid hydrolysis can be conducted at a temperature of from 30° C. to 200° C. In some further examples, acid hydrolysis can be conducted at a temperature of from 30° C. to 80° C., from 50° C. to 100° C., from 75° C. to 125° C., from 100° C. to 150° C., from 125° C. to 175° C., or from 150° C. to 200° C.

The incubation period can be a period that is sufficient at the concentration of acid and the temperature to hydrolyze the cells. For example, incubating the mixture at a higher temperature can result in the hydrolysis proceeding at a faster rate (i.e., requiring a shorter period of time for hydrolysis). Similarly, in some examples, incubating the mixture at a higher concentration of acid can result in the hydrolysis proceeding at a faster rate.

In some examples, hydrolysis of the cells in the biomass can be performed using enzymatic hydrolysis. For example, the biomass can be contacted with one or more enzymes under conditions that cause disruption of the cells. In some examples, the enzyme can be a protease. One non-limiting example of a protease that can be used for enzymatic hydrolysis of the biomass cells is ALCALASE 2.4L FG (Novozymes; Franklinton, N.C.). In some examples, the biomass can be washed prior to the enzymatic hydrolysis.

The microorganisms can typically be fermented to float in aqueous media. The fermentation media can be gravity settled in the fermenter and the media can be decanted or otherwise removed to provide the desired concentration of the biomass. Alternatively, the fermentation media can be concentrated by centrifugation to provide the desired concentration of the biomass. In some examples, the biomass can be contacted with one or more enzymes while the microorganisms are in the fermentation medium. Optimum temperature, time, pH and enzyme concentration depend on the specific enzyme employed. Enzymatic hydrolysis can be performed with or without the use of a surfactant.

Other methods of hydrolysis can also be employed. Non-limiting examples can include bead milling, sonication, rapid decompression, high-shear mechanical methods, homogenization, ultrasound, French press, cold-pressing, osmotic shock, heating, drying, pressure oscillation, solvent extraction, expression of an autolysis gene, the like, or a combination thereof. In some examples, hydrolysis of the biomass cells can be performed using a combination of chemical, enzymatic, and/or mechanical methods, either sequentially or concurrently.

Other methods or details related to hydrolysis can be found in U.S. Pat. No. 9,408,404 and U.S. Patent Publication Nos. 2022/0145211 and 2022/0154098, each of which is incorporated herein by reference in their entirety.

### Extraction

Lipids can be extracted from the biomass in a variety of ways. As used herein, the term “lipid” includes phospholipids, free fatty acids, esters of fatty acids, acylglycerols (e.g., triacylglycerols, diacylglycerols, monoacylglycerols), lysophospholipids, soaps, phosphatides, sterols, sterol esters, carotenoids, xanthophylls, hydrocarbons, and the like. The lipid-rich material that is extracted from the biomass can generally be referred to as a microbial oil, or as a crude oil.

Different types or components of the lipids can be extracted, depending on the extraction technique that is used. In some examples, the lipid can be isolated from a microorganism using standard techniques, without being subjected to further refinement or purification. In some examples, the lipid can be isolated using physical and/or mechanical extraction methods, such as, but not limited to, extraction via homogenization or pressing.

In other examples, the lipid can be extracted using solvent extraction. Where solvent extraction is used, a variety of organic solvents can be employed. Non-limiting examples can include hexane, isopropyl alcohol, methylene chloride, dodecane, methanol, ethylated oil, supercritical carbon dioxide, the like, or a combination thereof. Polar lipids (e.g., phospholipids) are generally extracted with polar solvents (e.g., chloroform/methanol). Non-polar lipids (e.g., triacylglycerols) are generally extracted with non-polar solvents (e.g., hexane). In some specific examples, where solvent extraction is used, the extraction solvent can include hexane.

In solvent extraction, the organic solvent and biomass can be mixed for a period of time suitable to extract lipids from the biomass. For example, the organic solvent and microorganisms can be mixed for a period of time of from 10 minutes to 2 hours or more. Subsequently, the lipid can be separated from the remaining components of the mixture by centrifugation.

In other examples, extraction can be performed using reduced amounts of organic solvent as compared to the amounts typically used to extract lipids from whole dry microbial cells. For example, the ratio of biomass to organic solvent needed to extract lipids from whole dry microbial cells is typically 1:4 or greater. Thus, the reduced amount of organic solvent can provide a ratio of biomass to organic solvent of less than 1:4.

In some specific examples, extraction can be performed in the absence of organic solvent. In some examples, “absence of organic solvent” means less than 0.5 wt. %, less than 0.4 wt. %, less than 0.3 wt. %, less than 0.2 wt. %, less than 0.1 wt. %, less than 0.05 wt. %, less than 0.01 wt. %, less than 0.005 wt. %, or 0 wt. % organic solvent based on the weight of the biomass.

Optionally, lipid extraction can be performed using an edible oil or biofuel. In some examples, the edible oil can be a plant oil, such as coconut oil, palm oil, canola oil, camelina oil, sunflower oil, soy oil, corn oil, olive oil, safflower oil, palm kernel oil, cottonseed oil, an alkylated derivative thereof, the like, or a combination thereof. As used herein, “biofuel” refers to any fuel, fuel additive, aromatic, and/or aliphatic compound derived from a biomass starting material. In some examples, biofuels can be derived from plant sources or microbial sources. Non-limiting examples of sources of biofuels can include microalgae, corn, switchgrass, sugarcane, sugarbeet, rapeseed, soybean, or the like. As used herein, the term organic solvent does not include biofuels as that term is defined herein and does not include an edible oil.

Optionally, where an edible oil and/or biofuel is used to extract lipids from the biomass, the edible oil and/or biofuel is not removed from the lipid extract. A subsequent fractionation of the extracted oil, wherein the added oil or biofuel stays with only one of the oil fractions, is not considered removal of the edible oil or biofuel from the lipid extract. For example, after recovery, the crude oil may be combined with other edible oils or biofuels for use as, or incorporated into, one of more or the nutritional oil compositions described herein. The edible oil or biofuel may be added to the microbial oil during the extraction step as an alternative to, or in addition to, combining with the microbial oil after the recovery process. In some cases, adding the edible oil or biofuel during the extraction step can assist in the demulsification and separation of the microbial oil from the biomass.

In traditional methods that rely on organic solvent extraction to separate the microbial oil from the biomass, the organic solvent is removed from the microbial oil after recovery, although trace amounts of the organic solvent may be left behind. In the methods described herein, where an edible oil or biofuel is employed to assist with the microbial oil extraction, optionally at least 80 wt. % of the edible oil or biofuel added during the extraction step can remain with the extracted microbial oil and can be incorporated into the final nutritional oil composition. In other examples, at least 85 wt. %, at least 90 wt. %, at least 95 wt. %, at least 98 wt. %, or 100 wt. % of the edible oil or biofuel used in the extraction step can remain with the extracted microbial oil and can be incorporated into the final nutritional oil composition.

Alternatively, the microbial oil can be extracted using mechanical methods. For example, they hydrolyzed biomass can be centrifuged to separate the microbial oil from the other components. In some examples, the microbial oil can be included an upper layer of the centrifuged material and can be isolated by suction or decanting from the residual material.

Other methods and details related to extraction can be found in U.S. Pat. Nos. 10,385,289; 10,364,207; 10,342,772; 10,472,316; 11,124,736; 11,352,651; and 11,001,782 and U.S. Patent Publication Nos. 2019/0300818 and 2020/0231898, each of which is incorporated herein by reference in their entirety.

### Refining

The microbial oil isolated from the other components of the biomass mixture can be referred to as a crude oil, which can include the PUFAs produced by the microorganisms, as well as a variety of other components extracted with the PUFAs, such as sterols, other fatty acids, residual edible oils/biofuels employed in the extraction process, etc. In some examples, the nutritional oil compositions described herein can be or include a crude oil extracted from the biomass.

Optionally, the microbial oil can be subjected to further refining processes to produce a refined oil or a final oil. In some examples, the nutritional oil compositions described herein can be or include a refined microbial oil or a final microbial oil.

Refining of the crude oil can include any of a variety of standard vegetable oil refining steps, such as degumming, neutralization/caustic refining, bleaching, deodorization, winterization/dewaxing, etc. When performed, refining can remove a variety of impurities or minor components, such as, but not limited to, free fatty acids, water, impurities, colorants, phospholipids, minerals, carotenoids, sterols, tocopherols/tocotrienols, waxes, residual cell debris, residual solvent, etc.

In further detail, degumming can be performed to remove phospholipids/gums from the crude oil, as desired. Degumming can also help to minimize refining losses and to minimize decomposition of the oil, for example. In some examples, degumming can include water degumming and/or acid degumming.

Neutralization is typically performed after degumming to remove free fatty acids. Neutralization typically includes saponifying the free fatty acids using an alkaline solution and extracting the resulting soap in a water phase, which can be easily separated from the oil. Neutralization can also be used to remove phospholipids, oxidized components in the oil, metal ions, colorants (e.g., color pigments), insoluble impurities, etc.

Bleaching is typically performed after neutralization and can be used to remove colorants (e.g., color pigments) from the oil. Bleaching can be performed by adding bleaching clays or other suitable adsorbents to adsorb colorants present in the oil. The bleaching clay or other adsorbent can then be removed by filtration.

Winterization or dewaxing is typically performed after bleaching and can be employed to removed waxes or saturated fatty acids from the oil. The presence of saturated fatty acids can cause the oil to have a cloudy or hazy appearance. Typically, winterization can be performed by chilling the oil to precipitate saturated fatty acids from the oil. The precipitated solids can then be filtered and removed from the oil.

Other methods and details related to refining of the crude oils can be found in U.S. Pat. Nos. 7,419,596 and 11,672,258, each of which is incorporated herein by reference in their entirety.

### Compositions

The present disclosure describes a variety of compositions, including microbial oil compositions and nutritional oil compositions. Generally, microbial oil compositions include a biomass component (e.g., prior to separating the microbial oil produced by the microorganism from the biomass). Nutritional oil compositions generally refer to compositions including the microbial oil after it has been extracted and separated from the biomass (e.g., compositions that do not include biomass). Typically, the microbial oil compositions and the nutritional oil compositions described herein can include at least 50 wt. % microbial oil (i.e., oil produced by microorganisms) based on the total amount of lipid present in the microbial oil compositions or nutritional oil compositions. In other examples, the microbial oil compositions and nutritional oil compositions described herein can include at least 60 wt. %, at least 70 wt. %, at least 80 wt. %, at least 90 wt. %, at least 95 wt. %, at least 97 wt. %, at least 98 wt. %, or at least 99 wt. % microbial oil based on the total amount of lipid present in the microbial oil compositions or the nutritional oil compositions. In still further examples, the microbial oil compositions and the nutritional oil compositions described herein can include from 50 wt. %. to 100 wt. %, from 60 wt. % to 99 wt. %, from 70 wt. % to 97 wt. %, or from 80 wt. % to 95 wt. % microbial oil based on the total amount of lipid present in the microbial oil compositions or the nutritional oil compositions.

The microbial oil compositions can include various amounts of biomass, depending on how the microorganisms were cultivated and the duration of the cultivation. In some examples, the microbial oil compositions can include biomass in an amount of at least 20 g/L based on the total dry cell weight of the washed biomass per unit volume of the culture medium. In some further examples, the microbial oil compositions can include biomass in an amount of at least 20 g/L, at least 40 g/L, at least 60 g/L, at least 80 g/L, at least 90 g/L, or at least 100 g/L. In still further examples, the microbial oil compositions can include biomass in an amount of from 20 g/L to 60 g/L, from 40 g/L to 80 g/L from 60 g/L to 100 g/L, from 70 g/L to 110 g/L, from 80 g/L to 120 g/L, from 90 g/L to 130 g/L, or from 100 g/L to 150 g/L. The biomass may include lysed and/or non-lysed cells.

The microbial oil compositions can also include a lipid component produced by the microorganisms. The lipid component can also be referred to as a microbial oil or as fat produced by the microorganisms. The amount of the lipid component or microbial oil can depend on a variety of factors as described elsewhere herein. In some examples, the microbial oil compositions can include at least 45 wt. % microbial oil based on the total dry cell weight of the washed biomass. In additional examples, the microbial oil compositions can include at least 50 wt. %, at least 55 wt. %, or at least 60 wt. % microbial oil based on the total dry cell weight of the washed biomass. In still further examples, the microbial oil compositions can include from 40 wt. % to 55 wt. %, from 45 wt. % to 60 wt. %, from 50 wt. % to 65 wt. %, or from 55 wt. % to 70 wt. % microbial oil based on the total dry cell weight of the washed biomass. The wt. % of microbial oil in the microbial oil composition and the amounts and proportions of the various fatty acids in of the microbial oil in the microbial oil composition may be determined as described below or in any suitable manner by methods known to the skilled person. Preferably, as described below, the biomass can be analyzed using American Oil Chemists' Society (AOCS) Official Method Ce 1b-89 or equivalent. While AOCS Ce 1b-89 is specifically written for oils, it can also be used to measure total lipid in the biomass by increasing the saponification time from 5 minutes to 45 minutes, for example, to more completely lyse the cells and more fully extract the oil. As is known in the art, the referenced AOCS method can be used to quantify individual fatty acid methyl esters (FAMEs), which can then be summed to provide total oil content obtained from the biomass. As the microbial oil compositions and the nutritional oil compositions each include a microbial oil, all references herein to the microbial oil are considered to apply equally to the microbial oil compositions and the nutritional oil compositions, unless the context clearly implies otherwise. In some examples, the nutritional oil compositions described herein can be or include a crude microbial oil or a refined microbial oil. In some examples, the nutritional oil compositions can be or include a crude microbial oil. In some examples, the nutritional oil compositions can be or include a refined or finished microbial oil. In some examples, the nutritional oil compositions can include the microbial oil in an amount greater than or equal to 50 wt. %, greater than or equal to 60 wt. %, greater than or equal to 70 wt. %, greater than or equal to 80 wt. %, greater than or equal to 90 wt. %, or greater than or equal to 95 wt. % based on a total weight of the nutritional oil composition. In some specific examples, the nutritional oil composition is a microbial oil extracted from a microorganism biomass. In some additional examples, the nutritional oil composition is a refined microbial oil obtained by refining a microbial oil extracted from the microorganism biomass, wherein said refining includes one or more steps selected from the group consisting of degumming, neutralization (or caustic refining), bleaching, deodorization and winterization (or dewaxing). In still additional examples, the nutritional oil composition is a crude microbial oil extracted from the microorganism biomass without refining, wherein said refining includes one or more steps selected from the group consisting of degumming, neutralization (or caustic refining), bleaching, deodorization, and winterization (or dewaxing).

The microbial oils can include a variety of fatty acids. Typically, the total fatty acids in the microbial oils can be identified and quantified using GC-MS and GC-FID, respectively. For example, American Oil Chemists' Society (AOCS) Official Method Ce 1b-89 can be used to obtain Fatty Acid Methyl Ester (FAME) data on the microbial oils. As is well understood in the art, the FAME data reports the identified fatty acids in relative area % of all of the fatty acid peaks in the GC chromatogram. Wt. % or mg/g values can be calculated by comparing the peak area for a particular fatty acid against a peak area or standard curve obtained using one or more quantitative reference standards.

The microbial oil compositions and nutritional oil compositions described herein can include a microbial oil enriched in EPA. In some examples, the microbial oils or nutritional oil compositions described herein can include EPA and DHA at a weight ratio of greater than or equal to 0.5 EPA/DHA or greater than or equal to 0.75 EPA/DHA. Typically, the microbial oils or nutritional oil compositions described herein include EPA in an amount greater than or equal to an amount of DHA present in the oil compositions. For example, EPA and DHA can be present in the microbial oils or nutritional oil compositions at a weight ratio of greater than or equal to 1.0 EPA/DHA. In some further examples, EPA and DHA can be present in the microbial oils or nutritional oil compositions at a weight ratio of greater than or equal to 1.05 EPA/DHA, greater than or equal to 1.1 EPA/DHA, greater than or equal to 1.2 EPA/DHA, greater than or equal to 1.3 EPA/DHA, greater than or equal to 1.4 EPA/DHA, greater than or equal to 1.5 EPA/DHA, greater than or equal to 1.6 EPA/DHA, greater than or equal to 1.7 EPA/DHA, greater than or equal to 1.8 EPA/DHA, greater than or equal to 1.9 EPA/DHA, or greater than or equal to 2.0 EPA/DHA. In some specific examples, EPA and DHA are present in the microbial oils or nutritional oil compositions at a weight ratio of less than 2.4 EPA/DHA, less than 2.2 EPA/DHA, less than 2.0 EPA/DHA, less than 1.8 EPA/DHA, less than 1.7 EPA/DHA, or less than 1.6 EPA/DHA. In some additional examples, EPA and DHA can be present in the microbial oils or nutritional oil compositions at a weight ratio of from 1.0 or 1.05 or 1.1 to 1.4 EPA/DHA, from 1.2 to 1.6 EPA/DHA, from 1.4 to 1.8 EPA/DHA, from 1.6 to 2.0 EPA/DHA, from 1.8 to 2.2 EPA/DHA, or from 2.0 to 2.4 EPA/DHA.

In addition to having a high ratio of EPA to DHA, the microbial oils or nutritional oil compositions disclosed herein can also have a high EPA titer. In some examples, EPA can be present in the microbial oils or nutritional oil compositions in an amount greater than or equal to 150 mg/g based on a total weight of the microbial oil or nutritional oil composition. In additional examples, EPA can be present in the microbial oils or nutritional oil compositions in an amount greater than or equal to 175 mg/g, greater than or equal to 200 mg/g, greater than or equal to 225 mg/g, greater than or equal to 250 mg/g, greater than or equal to 275 mg/g, greater than or equal to 300 mg/g, greater than or equal to 325 mg/g, greater than or equal to 350 mg/g, greater than or equal to 375 mg/g, or greater than or equal to 400 mg/g based on the total weight of the microbial oil or nutritional oil composition. In still additional examples, EPA can be present in the microbial oils or nutritional oil compositions in an amount of from 150 mg/g to 350 mg/g, from 200 mg/g to 400 mg/g, from 250 mg/g to 450 mg/g, from 300 mg/g to 500 mg/g, from 350 mg/g to 550 mg/g, or from 400 mg/g to 600 mg/g based on the total weight of the microbial oil or nutritional oil composition.

The nutritional oil compositions described herein also typically include an amount of DHA. In some examples, EPA and DHA are present in the microbial oils or nutritional oil compositions in a combined amount of greater than or equal to 300 mg/g based on the total weight of the microbial oil or nutritional oil composition. In further examples, EPA and DHA can be present in the microbial oils or nutritional oil compositions in a combined amount of greater than or equal to 350 mg/g, greater than or equal 400 mg/g, greater than or equal 450 mg/g, greater than or equal 500 mg/g, greater than or equal 550 mg/g, or greater than or equal 600 mg/g based on the total weight of the microbial oil or nutritional oil composition. In still further examples, EPA and DHA can be present in the microbial oils or nutritional oil compositions in a combined amount of from 350 mg/g to 450 mg/g, from 400 mg/g to 500 mg/g, from 450 mg/g to 550 mg/g, from 500 mg/g to 600 mg/g, from 550 mg/g to 650 mg/g, or from 600 mg/g to 700 mg/g based on the total weight of the microbial oil or nutritional oil composition. In some additional examples, EPA and DHA are each individually present in the microbial oils or nutritional oil compositions in an amount of greater than or equal to 150 mg/g, greater than or equal 175 mg/g, greater than or equal 200 mg/g, or greater than or equal 225 mg/g based on the total weight of the microbial oil or nutritional oil composition.

In some examples, DHA can be present in the microbial oils or nutritional oil compositions in an amount greater than or equal to 100 mg/g based on the total weight of the microbial oil or nutritional oil composition. In additional examples, DHA can be present in the microbial oils or nutritional oil compositions in an amount greater than or equal to 125 mg/g, greater than or equal to 150 mg/g, greater than or equal to 175 mg/g, greater than or equal to 200 mg/g, greater than or equal to 225 mg/g, or greater than or equal to 250 mg/g based on the total weight of the microbial oil or nutritional oil composition. In some additional examples, DHA can be present in the microbial oils or nutritional oil compositions in an amount not greater than 300 mg/g (i.e., DHA can be present in the oil composition in an amount greater than 0 mg/g up to 300 mg/g) based on the total weight of the microbial oil or the nutritional oil composition. In still additional examples, DHA can be present in the microbial oils or nutritional oil compositions in an amount not greater than 275 mg/g, not greater than 250 mg/g, or not greater than 225 mg/g based on the total weight of the microbial oil or the nutritional oil composition.

As the nutritional oil compositions described herein are or include a microbial oil (i.e., an oil produced by a microorganism), the microbial oils and the nutritional oil compositions typically include a variety of lipids other than EPA and DHA. For example, the microorganisms used to produce the microbial oil may also produce other omega-3 polyunsaturated fatty acids (PUFAs) in addition to EPA and DHA. Other omega-3 PUFAs can include a-linolenic acid (18:3n-3), stearidonic acid (18:4n-3), eicosatetraenoic acid (20:4n-3), and docosapentaenoic acid (22:5n-3).

In some examples, the microbial oils or nutritional oil compositions described herein can include docosapentaenoic acid (DPAn-3). In some examples, DPAn-3 can be present in the microbial oils or nutritional oil compositions in an amount not greater than 100 mg/g based on the total weight of the microbial oil or nutritional oil composition (e.g., DPAn-3 can be present in an amount greater than 0 mg/g up to 100 mg/g). In other examples, DPAn-3 can be present in the microbial oils or nutritional oil compositions in an amount not greater than 90 mg/g, not greater than 80 mg/g, not greater than 70 mg/g, not greater than 60 mg/g, or not greater than 50 mg/g based on the total weight of the microbial oil or nutritional oil composition.

DPAn-3 can typically be present in the microbial oils or nutritional oil compositions in an amount less than an amount of DHA present in the microbial oil or nutritional oil composition. In some examples, DHA and DPAn-3 can be present in the microbial oils or nutritional oil compositions at a weight ratio of greater than or equal to 1.3 DHA/DPAn-3. In additional examples, DHA and DPAn-3 can be present in the microbial oils or nutritional oil compositions at a weight ratio of greater than or equal to 1.5 DHA/DPA n-3, greater than or equal to 2.0 DHA/DPA n-3, greater than or equal to 2.5 DHA/DPA n-3, greater than or equal to 3.0 DHA/DPA n-3, greater than or equal to 3.5 DHA/DPA n-3, greater than or equal to 4.0 DHA/DPA n-3, greater than or equal to 4.5 DHA/DPA n-3, or greater than or equal to 5.0 DHA/DPA n-3. In some further examples, DHA and DPAn-3 can be present in the microbial oils or nutritional oil compositions at a weight ratio of from 1.0 to 2.0 DHA/DPAn-3, from 1.5 to 2.5 DHA/DPAn-3, from 2.0 to 3.0 DHA/DPAn-3, from 2.5 to 3.5 DHA/DPAn-3, from 3.0 to 4.0 DHA/DPAn-3, from 3.5 to 4.5 DHA/DPAn-3, from 4.0 to 5.0 DHA/DPAn-3, from 4.5 to 5.5 DHA/DPAn-3, or from 5.0 to 6.0 DHA/DPAn-3.

The microbial oils or nutritional oil compositions can also include other omega-3 PUFAs, such as a-linolenic acid, stearidonic acid, and/or eicosatetraenoic acid. Typically, the microbial oils or nutritional oil compositions described herein can have relatively high amounts of total omega-3 PUFAs. In some examples, the microbial oils or nutritional oil compositions can include total omega-3 PUFAs (i.e., the sum total of EPA, DHA, DPAn-3, a-linolenic acid, stearidonic acid, and eicosatetraenoic acid) in an amount greater than or equal to 300 mg/g based on the total weight of the microbial oil or nutritional oil composition. In additional examples, the microbial oils or nutritional oil compositions can include total omega-3 PUFAs in an amount greater than or equal to 350 mg/g, greater than or equal to 400 mg/g, greater than or equal to 450 mg/g, greater than or equal to 500 mg/g, greater than or equal to 550 mg/g, or greater than or equal to 600 mg/g based on the total weight of the microbial oil or nutritional oil composition.

The microbial oils or nutritional oil compositions described herein can also include one or more omega-6 PUFAs. Non-limiting examples, of omega-6 PUFAs that can be included in the microbial oils or nutritional oil compositions described herein can include linoleic acid (18:2n-6), gamma-linoleic acid (18:3n-6), arachidonic acid (20:4n-6), and/or docosapentaenoic acid (22:5n-6).

In some examples, docosapentaenoic acid (DPAn-6) can be present in the microbial oils or nutritional oil compositions in an amount not greater than 30 mg/g based on the total weight of the microbial oil or nutritional oil composition (e.g., in an amount greater than 0 mg/g up to 30 mg/g). In additional examples, DPAn-6 can be present in the microbial oils or nutritional oil compositions in an amount not greater than 25 mg/g, not greater than 20 mg/g, not greater than 15 mg/g, or not greater than 10 mg/g based on the total weight of the microbial oil or nutritional oil composition.

DPAn-6 can typically be present in the microbial oils or nutritional oil compositions in an amount less than an amount of DPA-3 present in the microbial oils or nutritional oil compositions. In some examples, DPAn-6 and DPAn-3 can be present in the microbial oils or nutritional oil compositions at a weight ratio of at least 1.5 DPAn-3/DPAn-6. In additional examples, DPAn-6 and DPAn-3 can be present in the microbial oils or nutritional oil compositions at a weight ratio of at least 2.0 DPAn-3/DPAn-6, at least 2.5 DPAn-3/DPAn-6, at least 3.0 DPAn-3/DPAn-6, at least 3.5 DPAn-3/DPAn-6, at least 4.0 DPAn-3/DPAn-6, at least 4.5 DPAn-3/DPAn-6, or at least 5.0 DPAn-3/DPAn-6. In some further examples, DPAn-3 and DPAn-6 can be present in the microbial oils or nutritional oil compositions at a weight ratio of from 1.0 to 2.0 DPAn-3/DPAn-6, from 1.5 to 2.5 DPAn-3/DPAn-6, from 2.0 to 3.0 DPAn-3/DPAn-6, from 2.5 to 3.5 DPAn-3/DPAn-6, from 3.0 to 4.0 DPAn-3/DPAn-6, from 3.5 to 4.5 DPAn-3/DPAn-6, from 4.0 to 5.0 DPAn-3/DPAn-6, from 4.5 to 5.5 DPAn-3/DPAn-6, or from 5.0 to 6.0 DPAn-3/DPAn-6.

In some specific examples, the microbial oils or nutritional oil compositions described herein can include arachidonic acid (ARA). In some examples, the microbial oils or nutritional oil compositions described herein can include ARA in an amount not greater than 40 mg/g based on the total weight of the microbial oil or nutritional oil composition (e.g., ARA can be present in an amount greater than 0 mg/g up to 40 mg/g). In additional examples, the microbial oils or nutritional oil compositions described herein can include ARA in an amount not greater than 35 mg/g, not greater than 30 mg/g, not greater than 25 mg/g, or not greater than 20 mg/g based on the total weight of the microbial oil or nutritional oil composition.

Where present, ARA can typically be present in the microbial oils or nutritional oil compositions in an amount greater than an amount of DPAn-6 present in the microbial oils or nutritional oil compositions. In some examples, ARA and DPAn-6 can be present in the microbial oils or nutritional oil compositions at a weight ratio of at least 1.2 ARA/DPAn-6. In further examples, ARA and DPAn-6 can be present in the microbial oils or nutritional oil compositions at a weight ratio of at least 1.5 ARA/DPAn-6, at least 2.0 ARA/DPAn-6, at least 2.5 ARA/DPAn-6, or at least 3.0 ARA/DPAn-6. In some further examples, ARA and DPAn-6 can be present in the microbial oils or nutritional oil compositions at a weight ratio of from 1.0 to 2.0 ARA/DPAn-6, from 1.5 to 2.5 ARA/DPAn-6, from 2.0 to 3.0 ARA/DPAn-6, from 2.5 to 3.5 ARA/DPAn-6, or from 3.0 to 4.0 ARA/DPAn-6.

In some examples, the microbial oils or nutritional oil compositions disclosed herein can also include a variety of saturated fatty acids. One such example can include palmitic acid. In some examples, the microbial oils or nutritional oil compositions can include palmitic acid in amount greater than or equal to 30 mg/g based on the total weight of the microbial oil or nutritional oil composition. In some further examples, the microbial oils or nutritional oil compositions can include palmitic acid in an amount greater than or equal to 50 mg/g, greater than or equal to 70 mg/g, greater than or equal to 90 mg/g, greater than or equal to 110 mg/g, greater than or equal to 130 mg/g, greater than or equal to 150 mg/g, greater than or equal to 180 mg/g, or greater than or equal to 200 mg/g based on the total weight of the microbial oil or nutritional oil composition. In some additional examples, palmitic acid is present in the microbial oils or nutritional oil compositions in an amount of from 30 mg/g to 70 mg/g, from 50 mg/g to 90 mg/g, from 70 mg/g to 110 mg/g, from 90 mg/g to 130 mg/g, from 110 mg/g to 150 mg/g, from 130 mg/g to 170 mg/g, from 150 mg/g to 190 mg/g, from 170 mg/g to 210 mg/g, from 190 mg/g to 230 mg/g, or from 200 mg/g to 250 mg/g based on the total weight of the microbial oil or nutritional oil composition.

While some of the fatty acids described herein may be found in the microbial oils or nutritional oil compositions as free fatty acids, in many cases, the fatty acids can be bound to a glycerol backbone to form an acylglycerol, whether that be a triacylglycerol, a diacylglycerol, or a monoacylglycerol. The amount of a particular type of acylglycerol (e.g., the amount of triacylglycerols, for example) present in the microbial oils or nutritional oil compositions can be determined using an 1H-NMR.

Typically, the microbial oils or the nutritional oil compositions described herein can predominantly include triacylglycerols. For example, in some cases, the microbial oils or nutritional oil compositions disclosed herein can include greater than or equal to 70 wt. % triacylglycerols (triglycerides) based on the total weight of the microbial oil or nutritional oil composition. In other examples, the microbial oils or nutritional oil compositions disclosed herein can include greater than or equal to 80 wt. %, greater than or equal to 90 wt. %, or greater than or equal to 95 wt. % triacylglycerols (triglycerides) based on the total weight of the microbial oil or nutritional oil composition.

In some further examples, the microbial oils or nutritional oil compositions described herein can include an amount of diacylglycerols (diglycerides) and/or monoacylglycerols (monoglycerides). In some specific examples, the microbial oils or nutritional oil compositions disclosed herein can include less than or equal to 20 wt. %, less than or equal to 10 wt. %, less than or equal to 5 wt. %, less than or equal to 3 wt. %, or less than or equal to 2 wt. % diacylglycerols (diglycerides) based on the total weight of the microbial oil or nutritional oil composition. In some specific examples, the microbial oils or nutritional oil compositions disclosed herein can include diacylglycerols (diglycerides) in an amount up to 20 wt. % (i.e., an amount of diacylglycerols greater than 0 wt. % up to 20 wt. %), an amount up to 10 wt. %, an amount up to 5 wt. %, an amount up to 3 wt. %, or an amount up to 2 wt. % based on the total weight of the microbial oil or nutritional oil composition.

In some additional specific examples, the microbial oils or nutritional oil compositions can include less than or equal to 10 wt. %, less than or equal to 5 wt. %, less than or equal to 3 wt. %, less than or equal to 2 wt. %, or less than or equal to 1 wt. % monoacylglycerols (monoglycerides) based on the total weight of the microbial oil or nutritional oil composition. In still additional specific examples, the microbial oils or nutritional oil compositions can include monoacylglycerols (monoglycerides) in an amount up to 10 wt. % (i.e., an amount of monoacylglycerols greater than 0 wt. % up to 10 wt. %), an amount up to 5 wt. %, an amount up to 3 wt. %, an amount up to 2 wt. %, or an amount up to 1 wt. % based on the total weight of the microbial oil or nutritional oil composition.

Fatty acids can be bound to glycerol at one of three positions on the glycerol backbone to form an acylglycerol. The two terminal hydroxyls along the glycerol backbone are referred to as the sn-1 and sn-3 positions. The central hydroxyl is referred to as the sn-2 position. The amount of a particular fatty acid positioned at the sn-1 and sn-3 positions relative to the amount of the same fatty acid positioned at the sn-2 position can be determined using 13C-NMR (see, for example, Suarez et. al. (2010), 13C-NMR Regioisomeric Analysis of EPA and DHA in Fish Oil Derived Triacylglycerol Concentrates, J Am Oil Chem Soc, 87:1425-1433).

Typically, based on the total EPA present in the acylglycerol fraction or component of the microbial oils or the nutritional oil compositions, at least 50% of the EPA can be bound at the sn-1/sn-3 positions, based on the total EPA present in the acylglycerol fraction or component of the microbial oil or the nutritional oil composition. In some examples, at least 55%, at least 60%, or at least 65% of the EPA can be bound at the sn-1/sn-3 positions, based on the total EPA present in the acylglycerol fraction or component of the microbial oil or the nutritional oil composition. In some examples, from 50% to 60%, from 55% to 65%, or from 60% to 70% of EPA can be bound at the sn-1/sn-3 positions, based on the total EPA present in the acylglycerol fraction or component of the microbial oil or the nutritional oil composition.

Typically, at least 45% of the DHA can be found at the sn-2 position, based on the total DHA present in the acylglycerol fraction or component of the microbial oil or the nutritional oil composition. In some examples, at least 50%, at least 55%, or at least 60% of the DHA can be found at the sn-2 position, based on the total DHA present in the acylglycerol fraction or component of the microbial oil or the nutritional oil composition. In some examples, from 45% to 55%, from 50% to 60%, or from 55% to 65% of the DHA can be found at the sn-2 position, based on the total DHA present in the acylglycerol fraction or component of the microbial oil or the nutritional oil composition.

In some examples, the microbial oils or the nutritional oil compositions include relatively low levels of free fatty acids. The amount of free fatty acids present in the microbial oils or the nutritional oil compositions can be determined using AOCS Official Method Ca 5a-40. Typically, the microbial oils or the nutritional oil compositions can include less than 1 wt. % free fatty acids based on the total weight of the microbial oil or the nutritional oil composition. In other examples, the microbial oils or the nutritional oil compositions have a free fatty acid content of less than 0.5 wt. % or less than 0.1 wt. % based on the total weight of the microbial oil or the nutritional oil composition.

In some additional examples, the microbial oils or the nutritional oil compositions can include a sterol. Unless specified to the contrary, reference to sterols includes both sterols and their respective sterol esters. The amount of sterols present in the microbial oils or the nutritional oil compositions can be quantified by isolating the non-saponifiable portion (including sterols) from the microbial oils or the nutritional oil compositions using AOCS Official Method Ca 6a-40. Individual sterols can be identified by GC-MS and quantified against reference standards using GC-FID. In some examples, the microbial oils or the nutritional oil compositions can include a total sterol content of up to 50 mg/g based on the total weight of the microbial oil or the nutritional oil composition (e.g., an amount greater than 0 mg/g up to 50 mg/g). In some further examples, the microbial oils or the nutritional oil compositions can include a total sterol content of up to 40 mg/g, up to 30 mg/g, up to 20 mg/g, or up to 10 mg/g based on the total weight of the microbial oil or the nutritional oil composition.

In some specific examples, the microbial oils or the nutritional oil compositions can include stigmasterol in an amount greater than or equal to 0.01 mg/g based on the total weight of the microbial oil or the nutritional oil composition. In additional examples, the microbial oils or the nutritional oil compositions can include stigmasterol in an amount greater than or equal to 0.05 mg/g, greater than or equal to 0.1 mg/g, greater than or equal to 0.25 mg/g, or greater than or equal to 0.5 mg/g based on the total weight of the microbial oil or the nutritional oil composition. In some further examples, the oil compositions can include stigmasterol in an amount from 0.01 mg/g to 0.1 mg/g, from 0.05 mg/g to 0.25 mg/g, from 0.1 mg/g to 0.5 mg/g, or from 0.25 mg/g to 0.75 mg/g based on the total weight of the microbial oil or the nutritional oil composition.

In additional specific examples, the microbial oils or the nutritional oil compositions can include cholesterol in an amount not greater than 10 mg/g (i.e., an amount greater than 0 mg/g up to 10 mg/g) based on the total weight of the microbial oil or the nutritional oil composition. In still additional examples, the microbial oils or the nutritional oil compositions can include cholesterol in an amount not greater than 5 mg/g, not greater than 3 mg/g, or not greater than 1 mg/g based on the total weight of the microbial oil or the nutritional oil composition.

The nutritional oil compositions described herein can typically be produced, and optionally further refined/purified, in a manner to minimize impurities and other undesired compounds in the oil compositions. Typically, the oil compositions described herein can have a relatively low peroxide value. Peroxide value is a measurement of the primary oxidation of hydroxyl groups of unsaturated fats by molecular oxygen into hydroperoxides and peroxides. It is typically viewed as an indicator of freshness of an edible oil. Peroxide value (PV) can be measured using AOCS Official Method Cd 8-53. Typically, the oil compositions can have a peroxide value of less than 5 meq/kg based on the total weight of the microbial oil or the nutritional oil composition. In some further examples, the microbial oils or the nutritional oil compositions can have a peroxide value of less than 4 meq/kg, less than 3 meq/kg, less than 2 meq/kg, less than 1 meq/kg, or less than 0.5 meq/kg based on the total weight of the microbial oil or the nutritional oil composition.

In further examples, the microbial oils or the nutritional oil compositions described herein can also have a relatively low anisidine value. The anisidine value is a measurement of the secondary oxidation compounds, primarily of 2-alkenals and 2,4-alkadienals generated from hydroperoxide decomposition. Anisidine value is also generally viewed as an indicator of freshness of an edible oil. Anisidine value can be determined using AOCS Official Method Cd 18-90. Typically, the microbial oils or the nutritional oil compositions described herein can have an anisidine value (AV) of less than 20. In additional examples, the microbial oils or the nutritional oil compositions described herein can have an AV of less than 15, less than 12, less than 10, less than 8, or less than 5.

In additional examples, the microbial oils or the nutritional oil compositions can have low 3-monochloropropane diol (3-MCPD) and 2,3-epoxy-1-propanol (glycidol) content. As similarly noted elsewhere herein, when referring to 3-MCPD and/or glycidol, such references also include reference to their respective esters unless otherwise specified. 3-MCPD and glycidol content can be identified and quantified using GC-MS/MS. These compounds are considered toxic to the human body and have been shown to induce tumors in rodent studies. In some examples, the microbial oils or the nutritional oil compositions described herein than have a 3-MCPD content of less than 1 mg/kg based on the total weight of the microbial oil or the nutritional oil composition. In other examples, the microbial oils or the nutritional oil compositions can have a 3-MCPD content of less than 0.5 mg/kg, less than 0.1 mg/kg, or less than 0.05 mg/kg based on a total weight of the microbial oil or the nutritional oil composition. In some further examples, the microbial oils or the nutritional oil compositions can have a glycidol content of less than 1 mg/kg based on the total weight of the microbial oil or the nutritional oil composition. In still further examples, the microbial oils or the nutritional oil compositions described herein can have a glycidol content of less than 0.5 mg/kg, less than 0.1 mg/kg, or less than 0.05 mg/kg based on a total weight of the microbial oil or the nutritional oil composition.

In some examples, the microbial oil compositions or the nutritional oil compositions can include one or more additives. Non-limiting examples can include an antioxidant, an antimicrobial agent, a flavoring, an odor-masking agent, an edible oil and/or biofuel, the like, or combinations thereof.

A variety of antioxidants can be included in the nutritional or microbial oil composition. In some examples, the antioxidant can be or include a natural antioxidant, such as a tocopherol, a phospholipid, ascorbic acid/ascorbyl palmitate, phytic acid, a phenolic acid, citric acid, rosemary extract, the like, or a combination thereof. In some additional examples, the antioxidant can be or include a synthetic antioxidant, such as butylated hydroxyanisole (BHA), butylated hydroxytoluene (BHT), propyl gallate, tertiary butyl hydroquinone (TBHQ), the like, or a combination thereof. In some specific examples, the antioxidant in the oil composition can be or include a tocopherol, ascorbyl palmitate, rosemary extract, or a combination thereof.

Anti-microbial agents can also include a variety of natural and/or synthetic agents. Non-limiting examples of anti-microbial agents can include rosemary extract, lemon oil, tea tree oil, clove oil, thyme oil, citral, sodium diacetate, nisin, natamycin, pediocin, reuterin, the like, or a combination thereof.

A variety of flavorings may also be included in the nutritional or microbial oil compositions. The present disclosure is not particularly limited on the type of flavoring that may be employed in the oil compositions disclosed herein. Any suitable flavoring may be employed, such as essential oil flavorings, flavoring concentrates, flavoring extracts, the like, or combinations thereof.

A variety of odor masking agents may also be used. The nutritional or microbial oil compositions disclosed herein are also not particularly limited as to the type of odor masking agent employed and any suitable odor masking agent is considered within the scope of the present disclosure. In some specific examples, the odor masking agent may also be a flavoring.

The concentration of the omega-3 PUFAs and/or total fat content may vary to some degree from one fermentation batch to another. As such, an edible oil and/or biofuel may be added to the nutritional oil compositions to help standardize the amount of omega-3 PUFAs in different lots of the nutritional oil compositions or to help achieve a particular concentration of one or more omega-3 PUFAs in the nutritional oil compositions, for example. Various edible oils and biofuels are described elsewhere herein, and such edible oils and/or biofuels can also be used in this context. In some specific examples, a plant oil can be added the nutritional oil composition. In some examples, a second microbial oil can be added to the nutritional oil composition.

The nutritional oil compositions, or a concentrate thereof, produced according to the methods described herein can be utilized in a variety of applications, such as to exploit their biological or nutritional properties. Non-limiting examples of potential applications can include pharmaceuticals, food supplements, animal feed additives, cosmetics, and the like.

The nutritional or microbial oil compositions described herein can be incorporated into a variety of food or feed supplements. For example, the nutritional or microbial oils can be included in a variety of beverages such as milk, sports drinks, energy drinks, teas, juices, etc. The nutritional or microbial oils can also be incorporated into confections, such as jellies and biscuits, dairy products, processed food products, such as soft rice or porridge, infant formula, breakfast cereals, and a variety of other food products. Other non-limiting examples can include pet foods, such as cat foods, dog foods, poultry foods, fish foods (including aquaculture), etc.

In additional examples, the nutritional oil compositions, or a concentrate thereof, can be incorporated into a dietary supplement, such as a multivitamin, an omega fatty acid supplement, or other suitable dietary supplement.

In some examples, the nutritional oil composition, or a concentrate thereof, can be incorporated into a pharmaceutical composition. The pharmaceutical composition can be formulated with a pharmaceutically acceptable carrier to be administered to a subject as a suitable dosage form. In some examples, the pharmaceutical composition can be formulated as a liquid dosage form, such as a suspension, an elixir, an oil, a gel, a paste, or the like. In some additional examples, the pharmaceutical composition can be formulated as a solid dosage form, such as a powder, a tablet, a capsule, a gummy, a lozenge, or the like.

The nutritional oil compositions, or a concentrate thereof, can be used in a variety of other nutritional and/or pharmaceutical applications for humans and animals in addition to those specifically referenced herein.

### Concentration

In some examples, the microbial oils or nutritional oil compositions can be concentrated to produce a nutritional oil concentrate including a concentrated EPA product enriched in EPA as compared to the microbial oil or nutritional oil composition itself. In other words, the concentrate can have a higher concentration of EPA than was present prior to the concentration process.

In further detail, the microbial oils or the nutritional oil compositions including acylglycerols (acylglycerides) can be combined with a concentration adjuvant under suitable conditions to produce an EPA fraction and a residual fraction. In some examples, the EPA fraction can include an alkyl ester of EPA. In some additional examples, the EPA fraction can include an acylglycerol having EPA residues.

The concentration adjuvant can include any adjuvant or set of adjuvants suitable to interact with an acylglycerol to produce an EPA fraction and a residual fraction. In some examples, the concentration adjuvant can be or include an enzymatic catalyst, such as a lipase, that can preferentially cleave saturated fatty acids from the acylglycerols present in the microbial oil or nutritional oil composition. In this example, the EPA fraction can include acylglycerides having EPA residues and the residual fraction can include the saturated fatty acids and other residues cleaved by the enzymatic catalyst.

The saturated fatty acids and other residues cleaved by the enzymatic catalyst can be separated from the EPA fraction. In some examples, separation and recovery of the EPA fraction can include distillation (e.g., fractional distillation, wiped film distillation, or other suitable distillation technique), chromatography, the like, or a combination thereof. The recovered EPA fraction can include acylglycerides enriched in EPA residues due to the removal of the saturated fats and other residues cleaved by the enzymatic catalyst. This method of concentration leaves EPA as a residue on an acylglyceride, which can be beneficial from a bioavailability perspective. This also leaves the concentrate in a more natural state than the transesterification methods discussed herein. Thus, in this example, the concentrated EPA product can be or include a concentrated acylglycerol composition including acylglycerols enriched in EPA residues.

In some examples, the concentration adjuvant can be or include a transesterification reactant, such as an alcohol, which can be combined with an acylglycerol in the presence of an acid or a base to produce fatty acid alkyl esters and glycerol (e.g., by converting the individual fatty acid residues bound to the glycerol backbone to free fatty acid alkyl esters and glycerol via a transesterification reaction). In this example, the EPA fraction can include an alkyl ester of EPA and the residual fraction can include glycerol and various other alkyl esters.

Where an alcohol is used as the transesterification reactant, a variety of alcohols can be used, such as a C1-C6 alkyl alcohol, for example. Non-limiting examples of alcohols that can be used as transesterification reactants can be or include ethanol, methanol, n-propanol, iso-propanol, n-butanol, iso-butanol, sec-butanol, tert-butanol, n-pentanol, n-hexanol, the like, or a combination thereof. Similarly, a variety of acids or bases can be combined with the alcohol to facilitate the transesterification reaction.

The fatty acid alkyl ester fraction can be separated or fractionated into various fractions based on fatty acid alkyl ester chain length. The desired fraction(s) can be recovered using suitable techniques. In some examples, separation and recovery of the EPA fraction can include distillation (e.g., fractional distillation, wiped film distillation, and/or other suitable distillation technique), chromatography, the like, or a combination thereof.

In some specific examples, a fraction including lower-chain fatty acid alkyl esters (e.g., fatty acid alkyl esters having carbon chain lengths up to C18) can be largely separated and removed from the rest of the fatty acid alkyl ester component to leave a nutritional oil concentrate enriched in EPA alkyl ester content. Further, with the removal of lower chain fatty acid alkyl esters, other fatty acid alkyl esters having higher chain lengths (e.g., chain lengths greater than C18, for example) can also be concentrated in the remaining composition. Thus, in some examples, EPA, DHA, and other omega-3 fatty acids can be concentrated in the nutritional oil concentrate as compared to the oil compositions prior to concentration. In other examples, DHA and other longer-chain fatty acids can also be separated as a residual fraction from the EPA fraction.

The concentrate produced via transesterification may be considered a concentrated fatty acid alkyl ester composition enriched in EPA. Thus, in some cases, the concentrated EPA product can be or include the concentrated fatty acid alkyl ester composition. As such, in some specific examples, the concentrated EPA product can include EPA alkyl esters. In some examples, the concentrated EPA product can include EPA ethyl esters. In some additional examples, the concentrated EPA product can include DHA alkyl esters. In still additional examples, the concentrated EPA product can include DHA ethyl esters. In some examples, the DHA alkyl esters are also substantially separated from the EPA alkyl esters, such as to leave less than 15 wt. %, less than 12 wt. %, less than 10 wt. %, less than 8 wt. %, less than 5 wt. %, or less than 3 wt. % DHA alkyl esters, or DHA residues on an acylglyceride, in the concentrated EPA product based on a total weight of the concentrated EPA product. In other examples, the concentrated EPA product can include from 15 wt. % to 35 wt. %, or from 20 wt. % to 30 wt. % DHA alkyl esters, or DHA residues on an acylglyceride, in the concentrated EPA product.

However, fatty acid alkyl esters are generally considered less bioavailable than acylglycerols. Thus, in other examples, the concentrated fatty acid alkyl ester composition can be combined with glycerol and allowed to react under conditions to provide a re-esterified acylglycerol (acylglyceride) composition comprising acylglycerols enriched in EPA residues. As such, in some examples, the concentrated EPA product can be or include a concentrated acylglycerol composition including acylglycerols having EPA residues.

Any suitable method for converting the fatty acid alkyl esters in the concentrated fatty acid alkyl ester composition to acylglycerols may be used. In some examples, the fatty acid alkyl esters in the concentrated fatty acid alkyl ester composition can be converted to acylglycerols via enzyme-catalyzed glycerolysis, such as by using an immobilized lipase enzyme to re-esterify the glycerol backbone with the fatty acids present in the concentrated fatty acid alkyl ester composition as alkyl esters. Other suitable processes may also be employed to convert the fatty acid alkyl esters of the concentrated fatty acid alkyl ester composition to acylglycerols. It is further noted that the amount of triacylglycerols vs. other acylglycerols can be controlled based on the equivalent ratio of hydroxyl equivalents from the glycerol and alkyl ester equivalents combined (e.g., an excess of hydroxyl equivalents will tend to produce more di- and monoacylglycerides).

In some examples, the concentration adjuvant can include both an enzymatic catalyst and a transesterification reactant. For example, the enzymatic catalyst can be used in combination with a transesterification reactant, such as to improve the reaction rate and/or yield. For example, in some cases, the enzymatic catalyst may be used to first cleave and remove the saturated fats, followed by employing the transesterification reactant to perform a transesterification reaction to further concentrate the EPA as either a concentrated fatty acid alkyl ester composition, or a concentrated acylglyceride composition after re-esterification of the concentrated fatty acid alkyl ester composition.

Additional methods and details related to the concentration of PUFAs in a nutritional or microbial oil composition can be found in U.S. Patent Publications 2009/0023808 and 2019/0071618, each of which is incorporated herein by reference.

### Concentrates

The nutritional oil concentrates described herein can be or include a nutritional oil composition that has undergone a concentration process to form and/or include a concentrated EPA product enriched in EPA. In some examples, the concentrated EPA product can be a concentrated fatty acid alkyl ester composition including an alkyl ester of EPA, such as an ethyl ester of EPA. In other examples, the concentrated EPA product can be a concentrated acylglycerol composition including an acylglycerol having EPA residues (e.g., an acylglycerol that has been enzymatically modified to remove saturated fatty acids or an acylglycerol that has been re-esterified from the concentrated fatty acid alkyl ester composition, for example). Where the concentrated EPA product is a concentrated acylglycerol composition, the nutritional oil concentrate can typically include at least 50 wt. % triacylglycerols (triglycerides) based on a total weight of the nutritional oil concentrate. In additional examples, where the concentrated EPA product is a concentrated acylglycerol composition, the nutritional oil concentrate can include at least 60 wt. %, at least 70 wt. %, at least 80 wt. %, or at least 90 wt. % triacylglycerols (triglycerides) based on a total weight of the nutritional oil concentrate. In still additional examples, the nutritional oil concentrate can include from 50 wt. % to 100 wt. %, from 60 wt. % to 97 wt. %, from 70 wt. % to 95 wt. %, or from 80 wt. % to 90 wt. % triacylglycerols (triglycerides) based on a total weight of the nutritional oil concentrate.

Whether the concentrated EPA product is a concentrated alkyl ester composition or a concentrated acylglycerol composition, the nutritional oil concentrate can be enriched in EPA. Typically, the nutritional oil concentrates can include EPA in an amount of at least 450 mg/g based on the total weight of the nutritional oil concentrate. In still additional examples, EPA can be present in the nutritional oil concentrates in an amount of at least 500 mg/g, at least 550 mg/g, at least 600 mg/g, at least 650 mg/g, at least 700 mg/g, or at least 750 mg/g based on a total weight of the nutritional oil concentrate. In still additional examples, EPA can be present in the nutritional oil concentrates in an amount of from 450 mg/g to 550 mg/g, from 500 mg/g to 600 mg/g, from 550 mg/g to 650 mg/g, from 600 mg/g to 700 mg/g, from 650 mg/g to 750 mg/g, from 700 mg/g to 800 mg/g, or from 750 mg/g to 850 mg/g based on a total weight of the nutritional oil concentrate.

In some additional examples, the nutritional oil concentrates can further include DHA. In some examples, the nutritional oil concentrates can include no more than 100 mg/g DHA, such as from 10 mg/g to 100 mg/g, or from 25 mg/g to 75 mg/g DHA based on a total weight of the nutritional oil concentrate. In other examples, the nutritional oil concentrate can include from 100 mg/g to 400 mg/g, from 150 mg/g to 350 mg/g, or from 200 mg/g to 300 mg/g DHA based on a total weight of the nutritional oil concentrate.

Thus, the nutritional oil concentrates described herein can include relatively high amounts of omega-3 fatty acids, whether as alkyl esters or as acylglyceride residues. Typically, the nutritional oil concentrates described herein can include at least 700 mg/g omega-3 fatty acids based on a total weight of the nutritional oil concentrate. In further examples, the nutritional oil concentrates can include at least 750 mg/g, at least 775 mg/g, at least 800 mg/g, at least 825 mg/g, or at least 850 mg/g omega-3 fatty acids based on a total weight of the nutritional oil concentrate. In still further examples, the nutritional oil concentrates can include from 700 mg/g to 900 mg/g, from 750 mg/g to 875 mg/g, from 775 mg/g to 850 mg/g, or from 800 mg/g to 825 mg/g omega-3 fatty acids based on a total weight of the nutritional oil concentrates.

Due to the concentration process, the nutritional oil concentrates typically have reduced levels of C12 to C18 fatty acids and other lower molecular weight components. Thus, in some examples, the nutritional oil concentrate can include palmitic acid in an amount less than 50 mg/g, less than 40 mg/g, less than 30 mg/g, less than 20 mg/g, or less than 10 mg/g based on a total weight of the nutritional oil concentrate.

The nutritional oil concentrates described herein can typically also have reduced or no C20:1n-9 fatty acids. Typically, the nutritional oil concentrates can include less than 30 mg/g C20:1n-9 fatty acids based on the total weight of the nutritional lipid concentrate. In some examples, the nutritional oil concentrates can include less than 20 mg/g, less than 10 mg/g, less than 5 mg/g, less than 1 mg/g, or less than 0.5 mg/g C20:1n-9 fatty acids based on the total weight of the nutritional oil concentrate. In some specific examples, the nutritional oil concentrate includes no detectible amount of C20:1n-9 fatty acids.

### EXAMPLE EMBODIMENTS

Various compositions and methods are disclosed herein. To aid in the understanding of these compositions and methods, a number of non-limiting example embodiments are provided in the following clauses for illustration purposes only. The following example embodiments can be combined together in any suitable combination, unless the context clearly suggests otherwise.

Clause 1. A microbial oil composition comprising:
a biomass present in a culture medium optionally at a concentration of at least 20 g/L, at least 40 g/L, at least 60 g/L, at least 80 g/L, at least 90 g/L, or at least 100 g/L, wherein the biomass comprises a microbial oil optionally at a concentration of at least 35 wt. %, at least 40 wt. %, at least 45 wt. %, at least 50 wt. %, at least 55 wt. %, or at least 60 wt. % based on a total dry cell weight of the biomass, the microbial oil comprising:
   EPA and DHA present at a weight ratio of greater than or equal to 1.0 EPA/DHA.

Clause 2. A microbial oil composition, comprising:
a biomass present in a culture medium at a concentration of at least 20 g/L, at least 40 g/L, at least 60 g/L, at least 80 g/L, at least 90 g/L, or at least 100 g/L, wherein the biomass comprises a microbial oil, the microbial oil comprising:
   EPA in an amount of at least 300 mg/g based a total weight of the microbial oil,
   DHA present at a weight ratio with EPA of greater than or equal to 1.0 EPA/DHA, and
   optionally, DPA n-3, with the proviso that, if present, DPA n-3 is present at a weight ratio with DHA of at least 1.3 DHA/DPAn-3.

Clause 3. A microbial oil composition, comprising:
a biomass present in a culture medium, wherein the biomass comprises a microbial oil in an amount of at least 35 wt. %, at least 40 wt. %, at least 45 wt. %, at least 50 wt. %, at least 55 wt. %, or at least 60 wt. % based on a total dry cell weight of the biomass, the microbial oil comprising:
   EPA and DHA present at a weight ratio of greater than or equal to 1.05 EPA/DHA, and
   DPAn-3 and DPAn-6 present at a weight ratio of at least 1.5 DPAn-3/DPAn-6.

Clause 4. A microbial oil composition, comprising:
a biomass present in a culture medium at a concentration of at least 20 g/L, at least 40 g/L, at least 60 g/L, at least 80 g/L, at least 90 g/L, or at least 100 g/L, wherein the biomass comprises a microbial oil, the microbial oil comprising:
   EPA and DHA present at a weight ratio of greater than or equal to 1.2 EPA/DHA; and
   optionally, DPAn-3, with the proviso that, if present, DPA n-3 is present at a weight ratio with DHA of at least 1.3 DHA/DPAn-3.

Clause 5. A microbial oil composition, comprising:
a biomass present in a culture medium, wherein the biomass comprises a microbial oil in an amount of at least 35 wt. %, at least 40 wt. %, at least 45 wt. %, at least 50 wt. %, at least 55 wt. %, or at least 60 wt. % based on a total dry cell weight of the biomass, the microbial oil comprising:
   EPA in an amount greater than or equal to 150 mg/g based on the total weight of the microbial oil;
   DHA in an amount greater than or equal to 125 mg/g based on the total weight of the microbial oil; and
   DPAn-3 and DPAn-6 present at a weight ratio of at least 1.5 DPAn-3/DPAn-6,
   wherein EPA and DHA are present at a weight ratio of at least 0.75 EPA/DHA or at least 1.0 EPA/DHA.

Clause 6. A microbial oil composition, comprising:
a biomass present in a culture medium at a concentration of at least 20 g/L, at least 40 g/L, at least 60 g/L, at least 80 g/L, at least 90 g/L, or at least 100 g/L, wherein the biomass comprises a microbial oil, the microbial oil comprising:
   70 wt. % or greater of triglycerides based on a total weight of the microbial oil;
   EPA in an amount greater than or equal to 200 mg/g based on the total weight of the microbial oil;
   DHA present at a weight ratio with EPA of at least 1.2 EPA/DHA; and
   DPAn-6.

Clause 7. A microbial oil composition, comprising:
a biomass present in a culture medium at a concentration of at least 20 g/L, at least 40 g/L, at least 60 g/L, at least 80 g/L, at least 90 g/L, or at least 100 g/L, wherein the biomass comprises a microbial oil, the microbial oil comprising:
   omega-3 PUFAs in an amount of greater than or equal to 500 mg/g based on a total weight of the microbial oil; and
   EPA and DHA each present in the microbial oil at a weight ratio of greater than or equal to 1.2 EPA/DHA.

Clause 8. A method of producing a microbial oil, comprising:
cultivating a microorganism biomass in a culture medium;
inducing the microorganism to produce a lipid component enriched in EPA to form a microbial oil according to any one of clauses 1 to 7;
extracting the lipid component from the microorganism biomass to obtain the microbial oil; and
optionally, refining the microbial oil to reduce an amount of a free fatty acid, an impurity, a saturated fat or fatty acid, a colorant, or a combination thereof in the microbial oil.

Clause 9. A method of producing a microbial oil, comprising:
a. cultivating a microorganism biomass in a culture medium to a concentration of at least 20 g/L, at least 40 g/L, at least 60 g/L, at least 80 g/L, at least 90 g/L, or at least 100 g/L;
b. inducing the microorganism to produce a lipid component enriched in EPA;
c. extracting the lipid component from the microorganism biomass to obtain the microbial oil; and
d. optionally, refining the microbial oil to reduce an amount of a free fatty acid, an impurity, a saturated fat or fatty acid, a colorant, or a combination thereof in the microbial oil,
   wherein, at the conclusion of step c. and prior to the start of optional step d., the microbial oil comprises:
      EPA and DHA in a combined amount of greater than or equal to 300 mg/g based on a total weight of the microbial oil, wherein EPA and DHA are present at a weight ratio of greater than or equal to 1.0 EPA/DHA.

Clause 10. A method of producing a microbial oil, comprising
cultivating a microorganism biomass in a culture medium to a concentration of at least 20 g/L, at least 40 g/L, at least 60 g/L, at least 80 g/L, at least 90 g/L, or at least 100 g/L;
inducing the microorganism to produce a lipid component enriched in EPA;
extracting the lipid component from the microorganism biomass to obtain the microbial oil; and
optionally, refining the microbial oil to reduce an amount of a free fatty acid, an impurity, a saturated fat or fatty acid, a colorant, or a combination thereof in the microbial oil,
wherein the microbial oil comprises:
   EPA and DHA in a combined amount of greater than or equal to 300 mg/g based on a total weight of the microbial oil, wherein EPA and DHA are present at a weight ratio of greater than or equal to 1.0 EPA/DHA,
   palmitic acid in an amount greater than or equal to 30 mg/g based on the total weight of the microbial oil,
   stigmasterol, and
   optionally, DPA n-3, with the proviso that, if present, DPA n-3 is present at a weight ratio with DHA of at least 1.3 DHA/DPA n-3.

Clause 11. A method of producing a microbial oil, comprising
cultivating a microorganism biomass in a culture medium to a concentration of at least 20 g/L, at least 40 g/L, at least 60 g/L, at least 80 g/L, at least 90 g/L, or at least 100 g/L;
inducing the microorganism to produce a lipid component enriched in EPA;
extracting the lipid component from the microorganism biomass to obtain the microbial oil; and
optionally, refining the microbial oil to reduce an amount of a free fatty acid, an impurity, a saturated fat or fatty acid, a colorant, or a combination thereof in the microbial oil,
wherein the microbial oil comprises:
   EPA in an amount greater than or equal to 300 mg/g based on a total weight of the microbial oil,
   DHA in an amount greater than or equal to 125 mg/g based on the total weight of the microbial oil, and
   palmitic acid in an amount greater than or equal to 30 mg/g based on the total weight of the microbial oil,
   wherein EPA and DHA are present at a weight ratio of at least 0.75 EPA/DHA or at least 1.0 EPA/DHA.

Clause 12. A method of producing a microbial oil, comprising
cultivating a microorganism biomass in a culture medium to a concentration of at least 20 g/L, at least 40 g/L, at least 60 g/L, at least 80 g/L, at least 90 g/L, or at least 100 g/L;
inducing the microorganism to produce a lipid component enriched in EPA;
extracting the lipid component from the microorganism biomass to obtain the microbial oil; and
optionally, refining the microbial oil to reduce an amount of a free fatty acid, an impurity, a saturated fat or fatty acid, a colorant, or a combination thereof in the microbial oil,
wherein the microbial oil comprises:
   70 wt. % or greater of triglycerides based on a total weight of the microbial oil,
   EPA in an amount greater than or equal to 200 mg/g based on the total weight of the microbial oil,
   DHA present at a weight ratio with EPA of at least 1.2 EPA/DHA, and
   DPA n-6.

Clause 13. A nutritional oil composition, comprising the microbial oil of any one of the preceding clauses.

Clause 14. A nutritional oil composition, comprising:
EPA and DHA present at a weight ratio of greater than or equal to 0.75 EPA/DHA or greater than or equal to 1.0 EPA/DHA.

Clause 15. A nutritional oil composition, comprising:
EPA in an amount of at least 300 mg/g based on a total weight of the nutritional oil composition; and
DHA present at a weight ratio to EPA of greater than or equal to 1.0 EPA/DHA;
palmitic acid in an amount greater than or equal to 30 mg/g based on the total weight of the nutritional oil composition, and
stigmasterol,
wherein DHA and DPAn-3 are present at a weight ratio of at least 1.3 DHA/DPAn-3, and
wherein DPAn-3 and DPAn-6 are present at a weight ratio of at least 1.5 DPAn-3/DPAn-6.

Clause 16. A nutritional oil composition, comprising:
EPA and DHA present at a weight ratio of greater than or equal to 1.2 EPA/DHA;
palmitic acid in an amount greater than or equal to 30 mg/g based on the total weight of the nutritional oil composition,
stigmasterol, and
optionally, DPA n-3, with the proviso that, if present, DPA n-3 is present at a weight ratio with DHA of at least 1.3 DHA/DPA n-3,
wherein the nutritional oil composition has an anisidine value of less than 20.

Clause 17. A nutritional oil composition, comprising:
EPA and DHA in a combined amount of greater than or equal to 300 mg/g based on a total weight of the nutritional oil composition, wherein EPA and DHA are present at a weight ratio of greater than or equal to 1.0 EPA/DHA;
palmitic acid in an amount greater than or equal to 30 mg/g based on the total weight of the nutritional oil composition;
stigmasterol; and
optionally, DPA n-3, with the proviso that, if present, DPA n-3 is present at a weight ratio with DHA of at least 1.3 DHA/DPA n-3,
wherein the nutritional oil composition has a peroxide value of less than or equal to 5 meq/kg based on the total weight of the nutritional oil composition, and wherein the nutritional oil composition has an anisidine value of less than 20.

Clause 18. A nutritional oil composition, comprising:
EPA in an amount greater than or equal to 200 mg/g based on a total weight of the nutritional oil composition;
DHA in an amount greater than or equal to 125 mg/g based on the total weight of the nutritional oil composition;
palmitic acid in an amount greater than or equal to 30 mg/g based on the total weight of the nutritional oil composition;
stigmasterol; and
an antioxidant,
wherein EPA and DHA are present at a weight ratio of at least 1.0 EPA/DHA, and
wherein the nutritional oil composition has an anisidine value of less than 20.

Clause 19. A nutritional oil composition, comprising:
70 wt. % or greater of triglycerides based on a total weight of the nutritional oil composition;
EPA in an amount greater than or equal to 200 mg/g based on the total weight of the nutritional oil composition;
DHA present at a weight ratio with EPA of at least 0.75 EPA/DHA or at least 1.0 EPA/DHA;
stigmasterol; and
DPA n-6,
wherein the nutritional oil composition has a peroxide value of less than or equal to 5 meq/kg based on the total weight of the nutritional oil composition, and wherein the nutritional oil composition has an anisidine value of less than 20.

Clause 20. The nutritional oil composition according to any one of the preceding claims, wherein the nutritional oil composition comprises at least 50 wt. %, at least 60 wt. %, at least 70 wt. %, at least 80 wt. %, or at least 90 wt. % of a microbial oil extracted from a microorganism biomass.

Clause 21. The nutritional oil composition according to any one of the preceding claims, wherein the nutritional oil composition is a microbial oil extracted from a microorganism biomass.

Clause 22. The nutritional oil composition according to any one of the preceding claims, wherein the nutritional oil composition is a refined microbial oil obtained by refining a microbial oil extracted from the microorganism biomass, wherein said refining includes one or more steps selected from the group consisting of degumming, neutralization (or caustic refining), bleaching, deodorization and winterization (or dewaxing).

Clause 23. The nutritional oil composition according to claim 18, wherein the nutritional oil composition is a crude microbial oil extracted from the microorganism biomass without refining, wherein said refining includes one or more steps selected from the group consisting of degumming, neutralization (or caustic refining), bleaching, deodorization, and winterization (or dewaxing).

Clause 24. The microbial oil composition or the method of producing a microbial oil or the nutritional oil composition according to any one of the preceding clauses, wherein greater than or equal to 50 wt. %, greater than or equal to 60 wt. %, greater than or equal to 70 wt. %, greater than or equal to 80 wt. %, or greater than or equal to 90 wt. % of the EPA and/or DHA is produced from a single microorganism source.

Clause 25. The microbial oil composition or the method of producing a microbial oil or the nutritional oil composition according to any one of the preceding clauses, wherein the single microorganism source is a microorganism of the order Thraustochytriales.

Clause 26. The microbial oil composition or the method of producing a microbial oil or the nutritional oil composition according to any one of the preceding clauses, wherein the microorganism of the order Thraustochytriales is a microorganism of the genus Schizochytrium or the genus Thraustochytrium.

Clause 27. The microbial oil composition or the method of producing a microbial oil or the nutritional oil composition according to any one of the preceding clauses, wherein EPA and DHA are present in the microbial oil or the nutritional oil composition in a combined amount of greater than or equal to 300 mg/g, greater than or equal to 350 mg/g, greater than or equal to 400 mg/g, greater than or equal to 450 mg/g, greater than or equal to 500 mg/g, greater than or equal to 550 mg/g, or greater than or equal to 600 mg/g based on the total weight of the microbial oil or nutritional oil composition.

Clause 28. The microbial oil composition or the method of producing a microbial oil or the nutritional oil composition according to any one of the preceding clauses, wherein EPA and DHA are present in the microbial oil or nutritional oil composition in a combined amount of from 350 mg/g to 450 mg/g, from 400 mg/g to 500 mg/g, from 450 mg/g to 550 mg/g, from 500 mg/g to 600 mg/g, from 550 mg/g to 650 mg/g, or from 600 mg/g to 700 mg/g based on the total weight of the microbial oil or nutritional oil composition.

Clause 29. The microbial oil composition or the method of producing a microbial oil or the nutritional oil composition according to any one of the preceding clauses, wherein EPA and DHA are each present in the microbial oil or nutritional oil composition in an amount of greater than or equal to 150 mg/g, greater than or equal to 175 mg/g, greater than or equal to 200 mg/g, or greater than or equal to 225 mg/g based on a total weight of the microbial oil or nutritional oil composition.

Clause 30. The microbial oil composition or the method of producing a microbial oil or the nutritional oil composition according to any one of the preceding clauses, wherein EPA and DHA are present in the microbial oil or nutritional oil composition at a weight ratio of greater than or equal to 0.75 EPA/DHA, greater than or equal to 1.0 EPA/DHA, greater than or equal to 1.05 or 1.1 EPA/DHA, greater than or equal to 1.2 EPA/DHA, greater than or equal to 1.3 EPA/DHA, greater than or equal to 1.4 EPA/DHA, greater than or equal to 1.5 EPA/DHA, greater than or equal to 1.6 EPA/DHA, greater than or equal to 1.7 EPA/DHA, greater than or equal to 1.8 EPA/DHA, greater than or equal to 1.9 EPA/DHA, or greater than or equal to 2.0 EPA/DHA.

Clause 31. The microbial oil composition or the method of producing a microbial oil or the nutritional oil composition according to any one of the preceding clauses, wherein EPA and DHA are present in the microbial oil or nutritional oil composition at a weight ratio of from 0.75 or 1.0 to 1.4 EPA/DHA, from 1.2 to 1.6 or 1.7 EPA/DHA, from 1.4 to 1.8 EPA/DHA, from 1.6 to 2.0 EPA/DHA, from 1.8 to 2.2 EPA/DHA, or from 2.0 to 2.4 EPA/DHA.

Clause 32. The microbial oil composition or the method of producing a microbial oil or the nutritional oil composition according to any one of the preceding clauses, wherein EPA and DHA are present in the microbial oil or nutritional oil composition at a weight ratio of less than 1.7 EPA/DHA or less than 1.6 EPA/DHA.

Clause 33. The microbial oil composition or the method of producing a microbial oil or the nutritional oil composition according to any one of the preceding clauses, wherein EPA is present in the microbial oil or nutritional oil composition in an amount of greater than or equal to 150 mg/g, greater than or equal to 175 mg/g, greater than or equal to 200 mg/g, greater than or equal to 225 mg/g, greater than or equal to 250 mg/g, greater than or equal to 275 mg/g, greater than or equal to 300 mg/g, greater than or equal to 325 mg/g, greater than or equal to 350 mg/g, greater than or equal to 375 mg/g, or greater than or equal to 400 mg/g based on the total weight of the microbial oil or nutritional oil composition.

Clause 34. The microbial oil composition or the method of producing a microbial oil or the nutritional oil composition according to any one of the preceding clauses, wherein EPA is present in the microbial oil or nutritional oil composition in an amount from 150 mg/g to 350 mg/g, from 200 mg/g to 400 mg/g, from 250 mg/g to 450 mg/g, from 300 mg/g to 500 mg/g, from 350 mg/g to 550 mg/g, or from 400 mg/g to 600 mg/g based on the total weight of the microbial oil or nutritional oil composition.

Clause 35. The microbial oil composition or the method of producing a microbial oil or the nutritional oil composition according to any one of the preceding clauses, wherein DHA is present in the microbial oil or nutritional oil composition in an amount of greater than or equal to 100 mg/g, greater than or equal to 125 mg/g, greater than or equal to 150 mg/g, greater than or equal to 175 mg/g, greater than or equal to 200 mg/g, greater than or equal to 225 mg/g, or greater than or equal to 250 mg/g based on the total weight of the microbial oil or nutritional oil composition.

Clause 36. The microbial oil composition or the method of producing a microbial oil or the nutritional oil composition according to any one of the preceding clauses, wherein DHA is present in the oil composition in an amount not greater than 200 mg/g, not greater than 225 mg/g, not greater than 250 mg/g, not greater than 275 mg/g, or not greater than 300 mg/g based on the total weight of the microbial oil or nutritional oil composition.

Clause 37. The microbial oil composition or the method of producing a microbial oil or the nutritional oil composition according to any one of the preceding clauses, wherein DHA and DPAn-3 are present at a weight ratio of greater than or equal to 1.3 DHA/DPAn-3, greater than or equal to 1.5 DHA/DPAn-3, greater than or equal to 2.0 DHA/DPAn-3, greater than or equal to 2.5 DHA/DPAn-3, greater than or equal to 3.0 DHA/DPAn-3, greater than or equal to 3.5 DHA/DPAn-3, greater than or equal to 4.0 DHA/DPAn-3, greater than or equal to 4.5 DHA/DPAn-3, or greater than or equal to 5.0 DHA/DPAn-3.

Clause 38. The microbial oil composition or the method of producing a microbial oil or the nutritional oil composition according to any one of the preceding clauses, wherein DPAn-3 is present in the microbial oil or nutritional oil composition in an amount not greater than 100 mg/g, not greater than 90 mg/g, not greater than 80 mg/g, no greater than 70 mg/g, not greater than 60 mg/g, or not greater than 50 mg/g based on the total weight of the microbial oil or nutritional oil composition.

Clause 39. The microbial oil composition or the method of producing a microbial oil or the nutritional oil composition according to any one of the preceding clauses, wherein palmitic acid is present in the microbial oil or nutritional oil composition in an amount greater than or equal to 30 mg/g, greater than or equal to 50 mg/g, greater than or equal to 70 mg/g, greater than or equal to 90 mg/g, greater than or equal to 110 mg/g, greater than or equal to 130 mg/g, or greater than or equal to 150 mg/g based on the total weight of the microbial oil or nutritional oil composition.

Clause 40. The microbial oil composition or the method of producing a microbial oil or the nutritional oil composition according to any one of the preceding clauses, wherein palmitic acid is present in the microbial oil or nutritional oil composition in an amount of from 30 mg/g to 70 mg/g, from 50 mg/g to 90 mg/g, from 70 mg/g to 110 mg/g, from 90 mg/g to 130 mg/g, from 110 mg/g to 150 mg/g, from 130 mg/g to 170 mg/g, from 150 mg/g to 190 mg/g, from 170 mg/g to 210 mg/g, or from 190 mg/g to 230 mg/g based on the total weight of the microbial oil or nutritional oil composition.

Clause 41. The microbial oil composition or the method of producing a microbial oil or the nutritional oil composition according to any one of the preceding clauses, wherein stigmasterol is present in the microbial oil or nutritional oil composition in an amount of greater than or equal to 0.01 mg/g, greater than or equal to 0.05 mg/g, greater than or equal to 0.1 mg/g, greater than or equal to 0.25 mg/g, or greater than or equal to 0.5 mg/g based on the total weight of the microbial oil or nutritional oil composition.

Clause 42. The microbial oil composition or the method of producing a microbial oil or the nutritional oil composition according to any one of the preceding clauses, wherein the microbial oil or nutritional oil composition comprises cholesterol in an amount not greater than 10 mg/g, not greater than 5 mg/g, not greater than 3 mg/g, or not greater than 1 mg/g based on the total weight of the microbial oil or nutritional oil composition.

Clause 43. The microbial oil composition or the method of producing a microbial oil or the nutritional oil composition according to any one of the preceding clauses, wherein the microbial oil or nutritional oil composition comprises total sterols in an amount not greater than 50 mg/g, not greater than 40 mg/g, not greater than 30 mg/g, not greater than 20 mg/g, or not greater than 10 mg/g based on the total weight of the microbial oil or nutritional oil composition.

Clause 44. The microbial oil composition or the method of producing a microbial oil or the nutritional oil composition according to any one of the preceding clauses, wherein the microbial oil or nutritional oil composition comprises greater than or equal to 70 wt. %, greater than or equal to 80 wt. %, greater than or equal to 90 wt. %, or greater than or equal to 95 wt. % triglycerides based on the total weight of the microbial oil or nutritional oil composition.

Clause 45. The microbial oil composition or the method of producing a microbial oil or the nutritional oil composition according to any one of the preceding clauses, wherein the microbial oil or nutritional oil composition comprises less than or equal to 20 wt. %, less than or equal to 10 wt. %, less than or equal to 5 wt. %, less than or equal to 3 wt. %, or less than or equal to 2 wt. % diglycerides based on the total weight of the microbial oil or nutritional oil composition.

Clause 46. The microbial oil composition or the method of producing a microbial oil or the nutritional oil composition according to any one of the preceding clauses, wherein the microbial oil or nutritional oil composition comprises less than or equal to 10 wt. %, less than or equal to 5 wt. %, less than or equal to 3 wt. %, less than or equal to 2 wt. %, or less than or equal to 1 wt. % monoglycerides based on the total weight of the microbial oil or nutritional oil composition.

Clause 47. The microbial oil composition or the method of producing a microbial oil or the nutritional oil composition according to any one of the preceding clauses, further comprising DPAn-6.

Clause 48. The microbial oil composition or the method of producing a microbial oil or the nutritional oil composition according to clause 47, wherein DPAn-6 is present in the microbial oil or nutritional oil composition in an amount not greater than 30 mg/g, not greater than 25 mg/g, not greater than 20 mg/g, not greater than 15 mg/g, or not greater than 10 mg/g based on the total weight of the microbial oil or nutritional oil composition.

Clause 49. The microbial oil composition or the method of producing a microbial oil or the nutritional oil composition according to any one of the preceding clauses, wherein DPAn-3 and DPAn-6 are present in the microbial oil or the nutritional oil composition at a weight ratio of at least 1.5 DPAn-3/DPAn-6, at least 2.0 DPAn-3/DPAn-6, at least 2.5 DPAn-3/DPAn-6, at least 3.0 DPAn-3/DPAn-6, at least 3.5 DPAn-3/DPAn-6, at least 4.0 DPAn-3/DPAn-6, at least 4.5 DPAn-3/DPAn-6, or at least 5.0 DPAn-3/DPAn-6.

Clause 50. The microbial oil composition or the method of producing a microbial oil or the nutritional oil composition according to any one of the preceding clauses, wherein the microbial oil or the nutritional oil composition further comprises ARA in an amount not greater than 40 mg/g based on the total weight of the microbial oil or nutritional oil composition.

Clause 51. The microbial oil composition or the method of producing a microbial oil or the nutritional oil composition according to clause 50, wherein ARA and DPAn-6 are present in the microbial oil or the nutritional oil composition at a weight ratio of at least 1.2 ARA/DPAn-6, at least 1.5 ARA/DPAn-6, at least 2.0 ARA/DPAn-6, at least 2.5 ARA/DPAn-6, or at least 3.0 ARA/DPAn-6.

Clause 52. The microbial oil composition or the method of producing a microbial oil or the nutritional oil composition according to any one of the preceding clauses, wherein the microbial oil or nutritional oil composition comprises total omega-3 PUFAs in an amount greater than or equal to 300 mg/g, greater than or equal to 350 mg/g, greater than or equal to 400 mg/g, greater than or equal to 450 mg/g, greater than or equal to 500 mg/g, greater than or equal to 550 mg/g, or greater than or equal to 600 mg/g based on the total weight of the microbial oil or nutritional oil composition.

Clause 53. The microbial oil composition or the method of producing a microbial oil or the nutritional oil composition according to any one of the preceding clauses, wherein the microbial oil or nutritional oil composition has a peroxide value of less than 5 meq/kg, less than 4 meq/kg, less than 3 meq/kg, less than 2 meq/kg, less than 1 meq/kg, or less than 0.5 meq/kg based on the total weight of the microbial oil or nutritional oil composition.

Clause 54. The microbial oil composition or the method of producing a microbial oil or the nutritional oil composition according to any one of the preceding clauses, wherein the microbial oil or nutritional oil composition has an anisidine value of less than 20, less than 15, less than 12, less than 10, less than 8, or less than 5.

Clause 55. The microbial oil composition or the method of producing a microbial oil or the nutritional oil composition according to any one of the preceding clauses, wherein the microbial oil or nutritional oil composition has a 3-MCPD content of less than 1 mg/kg, less than 0.5 mg/kg, less than 0.1 mg/kg, or less than 0.05 mg/kg based on the total weight of the microbial oil or nutritional oil composition.

Clause 56. The microbial oil composition or the method of producing a microbial oil or the nutritional oil composition according to any one of the preceding clauses, wherein the microbial oil or nutritional oil composition has a glycidol content of less than 1 mg/kg, less than 0.5 mg/kg, less than 0.1 mg/kg, or less than 0.05 mg/kg based on the total weight of the microbial oil or nutritional oil composition.

Clause 57. The microbial oil composition or the method of producing a microbial oil or the nutritional oil composition according to any one of the preceding clauses, wherein the microbial oil or nutritional oil composition has a free fatty acid content of less than 1 wt %, less than 0.5 wt %, or less than 0.1 wt % based on the total weight of the microbial oil or nutritional oil composition.

Clause 58. The microbial oil composition or the method of producing a microbial oil or the nutritional oil composition according to any one of the preceding clauses, further comprising an antioxidant.

Clause 59. The microbial oil composition or the method of producing a microbial oil or the nutritional oil composition according to clause 59, wherein the antioxidant comprises a tocopherol, ascorbyl palmitate, rosemary extract, or a combination thereof.

Clause 60. A nutritional oil concentrate, comprising:
a concentrated EPA product having at least 450 mg/g EPA, produced by:
combining the nutritional oil composition or microbial oil according to any one of the preceding clauses with a concentration adjuvant under conditions to produce an EPA fraction and a residual fraction; and
removing at least a portion of the residual fraction to provide the concentrated EPA product.

Clause 61. The nutritional oil concentrate of clause 60, wherein the concentrated EPA product comprises a concentrated alkyl ester composition.

Clause 62. The nutritional oil concentrate of clause 60, wherein the concentrated EPA product comprises a concentrated acylglycerol composition.

Clause 63. The nutritional oil concentrate of clause 62, wherein the acylglycerol composition comprises at least 50 wt. %, at least 60 wt. %, at least 70 wt. %, at least 80 wt. %, or at least 90 wt. % triacylglycerols based on a total weight of the nutritional oil concentrate.

Clause 64. The nutritional oil concentrate according to any of clauses 60 to 63, wherein EPA is present in the nutritional oil concentrate in an amount of at least 500 mg/g, at least 550 mg/g, at least 600 mg/g, at least 650 mg/g, at least 700 mg/g, or at least 750 mg/g based on the total weight of the nutritional oil concentrate.

Clause 65. The nutritional oil concentrate according to any of clauses 60 to 64, wherein palmitic acid is present in the nutritional oil concentrate in an amount less than 50 mg/g, less than 40 mg/g, less than 30 mg/g, less than 20 mg/g, or less than 10 mg/g based on the total weight of the nutritional oil concentrate.

Clause 66. A nutritional oil concentrate, comprising:
at least 450 mg/g EPA based on the total weight of the nutritional oil concentrate;
at least 700 mg/g omega-3 fatty acids based on the total weight of the nutritional oil concentrate; and
less than 30 mg/g C20:1n-9 based on the total weight of the nutritional oil concentrate.

Clause 67. The nutritional oil concentrate of clause 66, wherein EPA is present in the nutritional oil concentrate in an amount of at least 500 mg/g, at least 550 mg/g, at least 600 mg/g, at least 650 mg/g, at least 700 mg/g, or at least 750 mg/g based on the total weight of the nutritional oil concentrate.

Clause 68. The nutritional oil concentrate of any one of clauses 66 and 67, wherein EPA comprises an EPA alkyl ester.

Clause 69. The nutritional oil concentrate of any one of clauses 66 and 67, wherein EPA comprises an EPA residue of an acylglyceride.

Clause 70. The nutritional oil concentrate of any one of clauses 66 to 69, comprising from 700 mg/g to 900 mg/g, from 750 mg/g to 875 mg/g, from 775 mg/g to 850 mg/g, or from 800 mg/g to 825 mg/g omega-3 fatty acids based on a total weight of the nutritional oil concentrate.

Clause 71. The nutritional oil concentrate of any one of clauses 66 to 70, comprising less than 50 mg/g, less than 40 mg/g, less than 30 mg/g, less than 20 mg/g, or less than 10 mg/g palmitic acid based on the total weight of the nutritional oil concentrate.

Clause 72. The nutritional oil concentrate of any one of clauses 66 to 71, comprising from 10 mg/g to 100 mg/g, or from 25 mg/g to 75 mg/g DHA based on a total weight of the nutritional oil concentrate.

Clause 73. The nutritional oil concentrate of any one of clauses 66 to 71, comprising from 100 mg/g to 400 mg/g, from 150 mg/g to 350 mg/g, or from 200 mg/g to 300 mg/g DHA based on a total weight of the nutritional oil concentrate.

Clause 74. A nutritional or pharmaceutical composition, comprising:
an amount of the microbial oil, the nutritional oil composition, or the nutritional oil concentrate according to any one of the preceding clauses; and
an acceptable nutritional or pharmaceutical carrier.

Clause 75. The nutritional or pharmaceutical composition of clause 74, wherein the nutritional or pharmaceutical composition is formulated as a liquid.

Clause 76. The nutritional or pharmaceutical composition of clause 75, wherein the liquid is a suspension, an emulsion, an oil, a gel, an elixir, or a paste.

Clause 77. The nutritional or pharmaceutical composition of clause 74, wherein the nutritional or pharmaceutical composition is formulated as a solid.

Clause 78. The nutritional or pharmaceutical composition of clause 77, wherein the solid is a food, a powder, a tablet, a capsule, a gummy, or a lozenge.

Clause 79. The method according to any one of clauses 8 to 12, wherein inducing the microorganism to produce a lipid component enriched in EPA comprises cultivating the microorganism in a culture medium having an average dissolved CO2 concentration of at least 100 ppm, at least 120 ppm, at least 140 ppm, at least 160 ppm, or at least 180 ppm.

Clause 80. The method according to any one of clauses 8 to 12 or 79, wherein inducing the microorganism to produce a lipid component enriched in EPA comprises cultivating the microorganism in a culture medium having a peak dissolved CO2 concentration of greater than 50 ppm, greater than 100 ppm, greater than 150 ppm, greater than 200 ppm, or greater than 250 ppm.

Clause 81. The method according to any one of clauses 8 to 12 or 79 to 80, wherein inducing the microorganism to produce a lipid component enriched in EPA comprises cultivating the microorganism at a Na:K weight ratio of from 1.5:1 to 6:1 or from 1.75:1 to 4:1 or from 2:1 to 3:1.

Clause 82. The method according to any one of clauses 8 to 12 or 79 to 81, wherein inducing the microorganism to produce a lipid component enriched in EPA comprises culturing the microorganism at a N:P weight ratio of from 7:1 to 9:1, from 8:1 to 10:1, from 9:1 to 11:1, from 10:1 to 12:1, or from 11:1 to 13:1.

Clause 83. The method according to any one of clauses 8 to 12 or 79 to 82, wherein inducing the microorganism to produce a lipid component enriched in EPA comprises culturing the microorganism at a temperature of from 18° C. to 28° C., from 20° C. to 25° C., from 18° C. to 22° C., or from 21° C. to 23° C.

Clause 84. The method according to any one of clauses 8 to 12 or 79 to 83, wherein inducing the microorganism to produce a lipid component enriched in EPA comprises culturing the microorganism at a pH of a pH of from 6.5 to 8.0, from 6.8 to 7.8, or from 7.0 to 8.5 and shifting the pH from a range of 6.5 to 7.5 to a higher range of 7.5 to 8.5 during cultivation.

Clause 85. A method of making a nutritional oil concentrate having at least 450 mg/g EPA, comprising:
combining a nutritional oil composition or microbial oil according to any one of clauses 1 to 59 with a concentration adjuvant under conditions to produce an EPA fraction and a residual fraction; and
removing at least a portion of the residual fraction to provide a concentrated EPA product.

Clause 86. The method of clause 85, wherein the concentration adjuvant comprises an enzymatic catalyst for removing a fatty acid residue from an acylglycerol.

Clause 87. The method of clause 86, wherein the EPA fraction comprises an acylglycerol having an EPA residue and the residual fraction comprises saturated fatty acids.

Clause 88. The method of clause 85, wherein the concentration adjuvant comprises a transesterification reactant.

Clause 89. The method of clause 87, wherein the EPA fraction comprises an alkyl ester of EPA and the residual fraction comprises glycerol.

Clause 90. The method of any one of clauses 85 to 89, further comprising performing enzyme-catalyzed glycerolysis to esterify EPA residues to glycerol and/or acylglycerol to provide the concentrated EPA product.

Clause 91. The method of any one of clauses 85 to 90, wherein the concentrated EPA product comprises at least 60 wt. %, at least 70 wt. %, at least 80 wt. %, or at least 90 wt. % triacylglycerols based on a total weight of the concentrated EPA product.

Clause 92. The method of any one of clauses 85 to 91, wherein the concentrated EPA product comprises EPA in an amount of at least 500 mg/g, at least 550 mg/g, at least 600 mg/g, at least 650 mg/g, at least 700 mg/g, or at least 750 mg/g based on the total weight of the concentrated EPA product.

Clause 93. The method of any one of clauses 85 to 92, wherein the concentrated EPA product comprises EPA in an amount of from 450 mg/g to 550 mg/g, from 500 mg/g to 600 mg/g, from 550 mg/g to 650 mg/g, from 600 mg/g to 700 mg/g, from 650 mg/g to 750 mg/g, from 700 mg/g to 800 mg/g, or from 750 mg/g to 850 mg/g based on the total weight of the concentrated EPA product.

### Experimental Examples

Having generally described the microbial oil compositions, nutritional oil compositions, nutritional oil concentrates, and associated methods, a further understanding can be obtained by reference to the following experimental examples provided herein. The following experimental examples are provided for purposes of illustration only and are not intended to be limiting. The analytical methods used to collect the data points in the following examples are already described above in the written description. The culture media used in the following experimental examples is described in Table 2 of U.S. Pat. No. 9,045,785, except as otherwise indicated in the individual examples. For convenience, this information is reproduced in Table 1 below.

TABLE 1
Culture Media

| Ingredient | Concentration | | Ranges |
|---|---|---|---|
| Na₂SO₄ | 8.8 | g/L | 0-25, 2-20, or 3-10 |
| NaCl | 0.625 | g/L | 0-25, 0.1-10, or 0.5-5 |
| KCl | 1.0 | g/L | 0-5, 0.25-3, or 0.5-2 |
| MgSO₄•7H₂O | 5.0 | g/L | 0-10, 2-8, or 3-6 |
| (NH₄)₂SO₄ | 0.42 | g/L | 0-10, 0.25-5, or 0.05-3 |
| CaCl₂ | 0.29 | g/L | 0.1-5, 0.15-3, or 0.2-1 |
| T 154 (yeast extract) | 1.0 | g/L | 0-20, 0.1-10, or 0.5-5 |
| KH₂PO₄ | 1.765 | g/L | 0.1-10, 0.5-5, or 1-3 |

Post Autoclave (Metals)

| | | | |
|---|---|---|---|
| Citric acid | 46.82 | mg/L | 0.1-5000, 10-3000, or 40-2500 |
| FeSO₄•7H₂O | 10.30 | mg/L | 0.1-100, 1-50, or 5-25 |
| MnCl₂•4H₂O | 3.10 | mg/L | 0.1-100, 1-50, or 2-25 |
| ZnSO₄•7H₂O | 9.3 | mg/L | 0.01-100, 1-50, or 2-25 |
| CoCl₂•6H₂O | 0.04 | mg/L | 0-1, 0.001-0.1, or 0.01-0.1 |
| Na₂MoO₄•2H₂O | 0.04 | mg/L | 0.001-1, 0.005-0.5, or 0.01-0.1 |
| CuSO₄•5H₂O | 2.07 | mg/L | 0.1-100, 0.5-50, or 1-25 |
| NiSO₄•6H₂O | 2.07 | mg/L | 0.1-100, 0.5-50, or 1-25 |

Post Autoclave (Vitamins)

| | | | |
|---|---|---|---|
| Thiamine | 9.75 | mg/L | 0.1-100, 1-50, or 5-25 |
| Ca½-pantothenate | 3.33 | mg/L | 0.1-100, 0.1-50, or 1-10 |
| Biotin | 3.58 | mg/L | 0.1-100, 0.1-50, or 1-10 |

Post Autoclave (Carbon)

| | | | |
|---|---|---|---|
| Glucose | 30.0 | g/L | 5-150, 10-100, or 20-50 |

Nitrogen Feed

| | | | |
|---|---|---|---|
| NH₄OH | 23.6 | mL/L | 0-150, 10-100, or 15-50 |

### Example 1

In this example, Schizochytrium species (ATCC PTA-10208) was cultivated in a 2000 L fermenter having a culture medium composition within the ranges specified in Table 1 above. In further detail, the total fermentation period or cultivation period was 7 days, including an approximately 60-hour growth phase followed by a production phase for the remainder of the 7-day period. The pH was adjusted up from 7 to 7.5 from the growth phase to the production phase. The culture media included an initial Na:K weight ratio of 2.25 Na:K at the beginning of the growth phase. The total N:P weight ratio (i.e., total added mass of N relative to the total added mass of P) added over the entire cultivation period was 9.04 N:P. The culture media was supplemented with dissolved CO2 to achieve an average dissolved CO2 level of 137 ppm for the entire cultivation period with a peak of 233 ppm dissolved CO2 occurring at approximately the transition from the growth phase to the production phase. A biomass concentration of 98 g/L was achieved at the end of the cultivation period, based on total washed dry cell weight of the biomass per unit volume of the culture media. The total lipid (microbial oil) content of the biomass was 63 wt. % at the end of the cultivation period, based on the total washed dry cell weight of the biomass. The microbial oil was extracted from the biomass using aqueous extraction techniques without the use of an organic solvent to obtain a crude microbial oil as described in U.S. Pat. No. 10,385,289. The crude microbial oil was further purified using standard refining, bleaching, winterization, and deodorization techniques to obtain a refined microbial oil. A standard antioxidant cocktail was added to the refined microbial oil. The refined microbial oil was analyzed to determine fatty acid content and other characteristics as reported in Tables 2 and 3.

TABLE 2
Fatty Acid Methyl Ester (FAME) Profile

| Fatty Acid | % Area |
|---|---|
| 12:0 (lauric acid) | 0.0 |
| 14:0 (myristic acid) | 2.6 |
| 16:0 (palmitic acid) | 28.3 |
| 18:0 (stearic acid) | 1.8 |
| 20:0 (arachidic acid) | 0.5 |
| 20:3n-3 | 0.9 |
| 20:4 (ARA) | 2.4 |
| 20:5 n-3 (EPA) | 33.8 |
| 22:5 n-6 (DPAn-6) | 0.8 |
| 22:5 n-3 (DPAn-3) | 3.1 |
| 22:6 n-3 (DHA) | 25.5 |

TABLE 3
Additional Analysis of Refined Microbial Oil

| [EPA] | 310 | mg/g |
|---|---|---|
| [DHA] | 218 | mg/g |
| EPA/DHA | 1.42 | |
| [DPAn-3] | 23.5 | mg/g |
| [DPAn-6] | 6.2 | mg/g |
| DHA/DPAn-3 | 9.27 | |
| DPAn-3/DPAn-6 | 3.8 | |
| [Total Omega 3 Fatty Acids] | 558 | mg/g |
| [ARA] | 18.2 | |
| ARA/DPAn-6 | 2.9 | |
| [Free Fatty Acids (FFA)] | 0.06 | wt. % |
| PV | <0.1 | meq/kg |

| | |
|---|---|
| AV | 4.6 |

### Example 2

In this example, Schizochytrium species (ATCC PTA-9695) was cultivated in a 10 L fermenter in a culture medium composition within the ranges specified in Table 1 above and at different levels of dissolved CO2. CO2 supplementation in the culture medium began at 60 hours after inoculation. The PH levels were maintained at 7.0+/−0.1. Test samples were taken at 153 log hours, 178 log hours, and 189 log hours, respectively, to determine the effect of dissolved CO2 in the culture medium on EPA levels produced by the microorganisms and EPA/DHA ratios in the total lipid. The results are depicted in Table 4 below.

TABLE 4
Effect of Dissolved CO₂ on EPA Production in ATCC PTA-9695

| | | | | | | |
|---|---|---|---|---|---|---|
| | Dissolved | | Total | | | |
| | CO₂ Average | Biomass | Lipid | DHA | EPA | EPA/ |
| Sample | (Peak) ppm | g/L | % | % | % | DHA |

Log Hour 153

| | | | | | | | |
|---|---|---|---|---|---|---|---|
| 1 (Control) | 26 | (29) | 98.4 | 56 | 63 | 7.52 | 0.116 |
| 2 | 76 | (102) | 90.6 | 54 | 49 | 17.67 | 0.353 |
| 3 | 120 | (166) | 85 | 51 | 39 | 29.26 | 0.737 |
| 4 | 166 | (236) | 58.3 | 34 | 42 | 22.11 | 0.512 |

Log Hour 178

| | | | | | | | |
|---|---|---|---|---|---|---|---|
| 1 (Control) | 26 | (29) | 112.2 | 57 | 65 | 7.3 | 0.111 |
| 2 | 76 | (102) | 104.9 | 55 | 50 | 17.78 | 0.345 |
| 3 | 120 | (166) | 98.1 | 50 | 39 | 30.18 | 0.749 |
| 4 | 166 | (236) | 72.2 | 35 | 38 | 25.66 | 0.656 |

Log Hour 189

| | | | | | | | |
|---|---|---|---|---|---|---|---|
| 1 (Control) | 26 | (29) | 99.8 | 60 | 65 | 7.23 | 0.109 |
| 2 | 76 | (102) | 97.1 | 61 | 51 | 17.84 | 0.343 |
| 3 | 120 | (166) | 83.7 | 54 | 39 | 31.68 | 0.795 |
| 4 | 166 | (236) | 62.2 | 41 | 37 | 27.34 | 0.728 |

As can be seen in Table 4, the control samples of ATCC PTA-9695 produce only approximately 7-8% EPA (% FAME) based on the total fatty acids present in the lipid. Increasing the dissolved CO2 levels above baseline conditions in the culture media increased the EPA production in Samples 2-4. Samples 2 and 3 each showed increases in EPA production of approximately 2× and 4×, respectively, while maintaining comparable total biomass production and total lipid production. However, Sample 4, with the greatest amount of dissolved CO2 in the culture medium, began to exhibit more substantial adverse effects on both biomass production and total lipid production. Furthermore, Sample 4 exhibited lower EPA production than Sample 3 despite having a higher dissolved CO2 content in the culture media. Thus, while some dissolved CO2 in the culture media can be beneficial to increase EPA production in microbial cells, too much dissolved CO2 can actually lead to adverse effects, such as reduced biomass production, reduced total lipid production, and comparatively lower levels of EPA than can be produced at somewhat lower dissolved CO2 concentrations.

### Example 3

In this example, Schizochytrium sp. was cultivated in a 10 L fermenter in a culture medium composition within the ranges specified in Table 1 above and at different temperatures to determine effect of temperature on EPA production. The PH levels were maintained at 7.0+/−0.1. The results are depicted in Table 5 below.

TABLE 5
Effect of Temperature on EPA Production in Schizochytrium sp.

| | Temperature | Biomass | Total Lipid | DHA | EPA | EPA/ |
|---|---|---|---|---|---|---|
| Sample | (° C.) | g/L | % | % | % | DHA |
| 1 | 22.5 | 147.5 | 67 | 49.2 | 19.0 | 0.39 |
| 2 | 24 | 143.4 | 66 | 50.6 | 16.2 | 0.32 |
| 3 | 25.5 | 134.1 | 61 | 53.6 | 12.15 | 0.23 |
| 4 | 27 | 85.2 | 43 | 57.6 | 8.07 | 0.14 |

As can be seen in Table 5, biomass production, total lipid production, and EPA production as a percentage (% FAME) of total fatty acids increased with decreasing temperature. Similarly, the ratio of EPA/DHA also increased with decreasing temperature.

### Example 4

In this example, Schizochytrium species (ATCC PTA-10208) was cultivated in 10 L fermenters having a culture medium composition within the ranges specified in Table 1 above. The control samples were cultured at baseline conditions including an average dissolved CO2 level of 85 ppm (111 ppm peak) and a culture temperature of 22.5° C. CO2 supplementation in the culture medium began at 60 hours after inoculation. Additionally, under baseline conditions for the control samples the pH was maintained at 7.0 until the nitrogen feed was ended, after which the pH was adjusted to 7.5. Further, at baseline conditions, the Na/K ratio in the culture medium was 2.9 and the N/P ratio in the culture medium was 10.7. Samples were cultivated for approximately 7 days. Test samples were taken at day 6 and at day 7. Table 6 below shows a variety of adjustments that were made to baseline conditions to determine the effects of these adjustments on EPA production.

TABLE 6
Effect of Various Conditions on
EPA Production in ATCC PTA-10208

| | | | | | | |
|---|---|---|---|---|---|---|
| | | | Total | | | |
| | | Biomass | Lipid | DHA | EPA | EPA/ |
| Sample | Altered Conditions | g/L | % | % | % | DHA |

Day 6

| | | | | | | |
|---|---|---|---|---|---|---|
| 1 | Control | 150 | 61 | 41.8 | 21.0 | 0.50 |
| 2 | Na/K = 2.11, | 110 | 59 | 29.2 | 31.2 | 1.07 |
| | N/P = 12.3, | | | | | |
| | dCO2 = 105 ppm | | | | | |
| | (144 ppm peak) | | | | | |
| 3 | Reduce temperature | 116 | 57 | 33.3 | 29.3 | 0.88 |
| | to 20° C. after N feed | | | | | |
| | stopped | | | | | |
| 4 | Adjust pH to 8 after N | 115 | 55 | 29.2 | 29.9 | 1.02 |
| | feed stopped | | | | | |
| 5 | Adjust pH to 8 after N | 80 | 42 | 33.3 | 28.8 | 0.86 |
| | feed stopped, | | | | | |
| | dCO2 = 115 ppm | | | | | |
| | (144 ppm peak) | | | | | |
| 6 | Na/K = 2.11, | 110 | 55 | 20.9 | 35.9 | 1.72 |
| | N/P = 12.3, | | | | | |
| | dCO2 = 160 ppm | | | | | |
| | (215 ppm peak) | | | | | |

Day 7

| | | | | | | |
|---|---|---|---|---|---|---|
| 1 | Control | 157 | 65 | 44.3 | 19.3 | 0.44 |
| 2 | Na/K = 2.11, | 122 | 62 | 32.5 | 29.6 | 0.91 |
| | N/P = 12.3, | | | | | |
| | dCO2 = 105 ppm | | | | | |
| | (144 ppm peak) | | | | | |
| 3 | Reduce temperature | 134 | 62 | 37.7 | 26.9 | 0.71 |
| | to 20° C. after N feed | | | | | |
| | stopped | | | | | |
| 4 | Adjust pH to 8 after N | 131 | 59 | 33.7 | 27.6 | 0.82 |
| | feed stopped | | | | | |
| 5 | Adjust pH to 8 after N | 87 | 42 | 32.7 | 29.0 | 0.89 |
| | feed stopped, | | | | | |
| | dCO2 = 115 ppm | | | | | |
| | (144 ppm peak) | | | | | |
| 6 | Na/K = 2.11, | 130 | 60 | 24.9 | 34.1 | 1.37 |
| | N/P = 12.3, | | | | | |
| | dCO2 = 160 ppm | | | | | |
| | (215 ppm peak) | | | | | |

### Example 5

In this example, Schizochytrium species (ATCC PTA-10208) was cultivated in 10 L fermenters having a culture medium composition within the ranges specified in Table 1 above. The Na:K ratio and N:P ratio were adjusted for each of the samples to determine the impact on EPA production and EPA/DHA ratio. The control sample had a Na:K ratio of approximately 5, a N:P ratio of approximately 15, and an average dissolved CO2 content of approximately 75 ppm with a peak CO2 content of approximately 123 ppm. Samples were cultivated at a temperature of 22.5° C. and a pH of 7. The altered conditions as compared to the control and effects on biomass production, total lipid production, EPA and DHA content are presented in Table 7 below. Table 8 presents additional analytical results for the same set of samples.

TABLE 7
Effect of Various Conditions on
EPA Production in ATCC PTA-10208

| | Altered | Biomass | Total Lipid | DHA | EPA | EPA/ |
|---|---|---|---|---|---|---|
| Sample | Conditions | g/L | % | mg/g | mg/g | DHA |
| 1 | Control | 74.1 | 61.5 | 386.9 | 183.0 | 0.47 |
| 2 | Na:K = 2.2 | 90.9 | 60.9 | 397.8 | 191.4 | 0.48 |
| 3 | N:P = 10 | 105.6 | 61.8 | 397.0 | 142.9 | 0.36 |
| 4 | Na:K = 2.2, | 109.7 | 64.4 | 373.0 | 202.9 | 0.54 |
| | N:P = 10 | | | | | |

TABLE 8
Effect of Various Conditions on Lipid
Production in ATCC PTA-10208

| | Sam- | Sam- | Sam- | Sam- |
|---|---|---|---|---|
| | ple 1 | ple 2 | ple 3 | ple 4 |
| % TAG | 92.7 | 92.8 | 92.5 | 93.1 |
| % DAG | 3.9 | 3.6 | 4.7 | 3.5 |
| % MAG | 0.2 | 0.2 | 0.2 | 0.2 |
| % Total Sterols | 0.9 | 1.0 | 0.6 | 0.8 |
| [Stigmasterol] mg/g | 6.54 | 6.96 | 5.09 | 5.35 |
| [Cholesterol] mg/g | 1.28 | 1.30 | 0.91 | 1.17 |
| [12:0 (lauric acid)] mg/g | 0.96 | 0.95 | 1.08 | 0.74 |
| [14:0 (myristic acid)] mg/g | 14.6 | 15.0 | 17.7 | 12.4 |
| [16:0 (palmitic acid)] mg/g | 209.9 | 213.2 | 249.3 | 205.5 |
| [18:0 (stearic acid)] mg/g | 14.6 | 14.0 | 15.9 | 16.0 |
| [20:4 (ARA)] mg/g | 18.2 | 18.7 | 17.6 | 19.6 |
| [22:5 n-6 (DPAn-6)] mg/g | 18.2 | 18.4 | 21.0 | 17.3 |
| [22:5 n-3 (DPAn-3)] mg/g | 35.8 | 47.0 | 15.6 | 38.7 |
| [Total Omega 3] mg/g | 605.7 | 636.2 | 555.5 | 614.5 |
| DHA/DPAn-3 | 10.8 | 8.5 | 25.4 | 9.7 |
| DPAn-3/DPAn-6 | 2.0 | 2.6 | 0.7 | 2.2 |
| ARA/DPAn-6 | 1.0 | 1.0 | 0.8 | 1.1 |

### Example 6

In this example, Schizochytrium species (ATCC PTA-10208) was cultivated in 10 L fermenters having a culture medium composition within the ranges specified in Table 1 above. Based on the results of Example 5, a new set of conditions was trialed starting with a baseline control sample cultivated under conditions equivalent to Sample 4 of Example 5, except that the average dissolved CO2 content was increased to approximately 172 ppm and the peak dissolved CO2 content was increased to approximately 262 ppm. The altered conditions as compared to the control and effects on biomass production, total lipid production, EPA and DHA content are presented in Table 9 below. Table 10 presents additional analytical results for the same set of samples.

TABLE 9
Effect of Various Conditions on
EPA Production in ATCC PTA-10208

| | | | Total | | | |
|---|---|---|---|---|---|---|
| | | Biomass | Lipid | DHA | EPA | EPA/ |
| Sample | Altered Conditions | g/L | % | mg/g | mg/g | DHA |
| 1 | Control | 97.5 | 58.1 | 233.4 | 310.1 | 1.33 |
| 2 | Na:K = 4.3, | 85.8 | 54.9 | 227.8 | 295.9 | 1.30 |
| | N:P = 12 | | | | | |
| 3 | Temp. = 20.5° C. | 89.7 | 54.1 | 177.5 | 380.4 | 2.14 |
| 4 | Reduce dO₂ from 10% | 98.6 | 57.9 | 210.8 | 318.3 | 1.51 |
| | to 5% after N feed | | | | | |
| | stopped | | | | | |
| 5 | Adjust pH from 7 to 8 | 59.3 | 44.1 | 197.1 | 319.9 | 1.51 |
| | after N feed stopped | | | | | |

TABLE 10
Effect of Various Conditions on Lipid
Production in ATCC PTA-10208

| | Sam- | Sam- | Sam- | Sam- | Sam- |
|---|---|---|---|---|---|
| | ple 1 | ple 2 | ple 3 | ple 4 | ple 5 |
| % TAG | 95.4 | 92.7 | 95.2 | 95.7 | 92.9 |
| % DAG | 2.7 | 2.5 | 2.4 | 2.0 | 4.0 |
| % MAG | 0.2 | 0.2 | 0.2 | 0.2 | 0.3 |
| % Total Sterols | 1.0 | 1.1 | 1.0 | 0.9 | 1.5 |
| [Stigmasterol] mg/g | 6.6 | 7.3 | 7.0 | 4.2 | 9.4 |
| [Cholesterol] mg/g | 1.0 | 1.1 | 1.2 | 0.8 | 1.3 |
| [12:0 (lauric acid)] mg/g | 1.5 | 1.2 | 1.6 | 1.6 | 1.5 |
| [14:0 (myristic acid)] mg/g | 21.2 | 16.8 | 20.0 | 20.9 | 19.7 |
| [16:0 (palmitic acid)] mg/g | 218.1 | 221.4 | 217.3 | 215.9 | 206.0 |
| [18:0 (stearic acid)] mg/g | 14.8 | 17.2 | 15.4 | 15.3 | 18.6 |
| [20:4 (ARA)] mg/g | 22.0 | 21.4 | 18.7 | 20.4 | 21.6 |
| [22:5 n-6 (DPAn-6)] mg/g | 6.6 | 5.9 | 3.6 | 5.6 | 4.1 |
| [22:5 n-3 (DPAn-3)] mg/g | 51.5 | 48.2 | 40.8 | 72.5 | 56.1 |
| [Total Omega 3] mg/g | 595.1 | 571.8 | 598.7 | 601.5 | 573.0 |
| DHA/DPAn-3 | 4.5 | 4.7 | 4.4 | 2.9 | 3.5 |
| DPAn-3/DPAn-6 | 7.8 | 8.2 | 11.3 | 12.9 | 13.7 |
| ARA/DPAn-6 | 3.3 | 3.6 | 5.2 | 3.6 | 5.3 |

Sample 3 was further processed using standard refining, bleaching, winterization, and deodorization (RBWD) techniques to produce a refined/final oil. The refined oil was analyzed in the same manner as the crude oil above. The results are presented in Table 11 below.

TABLE 11
Effect of Various Conditions on Final/Refined
Oil Based on Sample 3

| | Sample 3 |
|---|---|
| % TAG | 97.0 |
| % DAG | 1.1 |
| % MAG | 0.2 |
| % Total Sterols | 0.5 |
| [Stigmasterol] mg/g | 2.8 |
| [Cholesterol] mg/g | 0.4 |
| [12:0 (lauric acid)] mg/g | 1.4 |
| [14:0 (myristic acid)] mg/g | 18.8 |
| [16:0 (palmitic acid)] mg/g | 204.1 |
| [18:0 (stearic acid)] mg/g | 13.7 |
| [EPA] mg/g | 364.7 |
| [DHA] mg/g | 160.8 |
| EPA/DHA | 2.3 |
| [20:4 (ARA)] mg/g | 18.4 |
| [22:5 n-6 (DPAn-6)] mg/g | 1.2 |
| [22:5 n-3 (DPAn-3)] mg/g | 38.9 |
| [Total Omega 3] mg/g | 564.5 |
| DHA/DPAn-3 | 4.1 |
| DPAn-3/DPAn-6 | 32.4 |
| ARA/DPAn-6 | 15.3 |

### Example 7

Schizochytrium sp. was cultivated in 10 L fermenters under the same conditions as Example 6 to evaluate the response of a strain believed to be a low EPA producer to those same conditions. The altered conditions as compared to the control and effects on biomass production, total lipid production, EPA and DHA content are presented in Table 12 below. Table 13 presents additional analytical results for the same set of samples.

TABLE 12
Effect of Various Conditions on EPA
Production in Schizochytrium sp.

| | | | Total | | | |
|---|---|---|---|---|---|---|
| | | Biomass | Lipid | DHA | EPA | EPA/ |
| Sample | Altered Conditions | g/L | % | mg/g | mg/g | DHA |
| 1 | Control | 96.9 | 58.4 | 391.1 | 215.0 | 0.55 |
| 2 | Na:K = 4.3 | 89.7 | 54.0 | 340.3 | 274.8 | 0.81 |
| | N:P = 12 | | | | | |
| 3 | Temp. = 20.5° C. | 99.5 | 54.9 | 362.0 | 255.5 | 0.71 |
| 4 | Reduce dO₂ from 10% | 95.4 | 59.6 | 334.9 | 290.0 | 0.87 |
| | to 5% after N feed | | | | | |
| | stopped | | | | | |
| 5 | Adjust pH from 7 to 8 | 51.6 | 36.5 | 281.4 | 328.9 | 1.17 |
| | after N feed stopped | | | | | |

TABLE 13
Effect of Various Conditions on Lipid
Production in Schizochytrium sp.

| | Sam- | Sam- | Sam- | Sam- | Sam- |
|---|---|---|---|---|---|
| | ple 1 | ple 2 | ple 3 | ple 4 | ple 5 |
| % TAG | 95.1 | 94.9 | 95.0 | 94.7 | 92.0 |
| % DAG | 2.2 | 2.4 | 2.0 | 2.6 | 3.4 |
| % MAG | 0.2 | 0.2 | 0.2 | 0.2 | 0.2 |
| % Total Sterols | 0.8 | 0.7 | 0.7 | 0.8 | 1.3 |
| [Stigmasterol] mg/g | 5.4 | 5.2 | 4.8 | 5.3 | 8.1 |
| [Cholesterol] mg/g | 0.9 | 0.9 | 0.8 | 1.1 | 1.4 |
| [12:0 (lauric acid)] mg/g | 1.5 | 1.2 | 1.4 | 1.4 | 1.1 |
| [14:0 (myristic acid)] mg/g | 21.7 | 18.0 | 20.5 | 21.2 | 14.4 |
| [16:0 (palmitic acid)] mg/g | 216.4 | 177.4 | 206.3 | 180.5 | 139.5 |
| [18:0 (stearic acid)] mg/g | 10.9 | 11.0 | 10.9 | 10.1 | 12.9 |
| [20:4 (ARA)] mg/g | 23.7 | 31.6 | 22.6 | 27.6 | 33.4 |
| [22:5 n-6 (DPAn-6)] mg/g | 17.3 | 14.8 | 12.5 | 11.3 | 8.2 |
| [22:5 n-3 (DPAn-3)] mg/g | 16.7 | 23.1 | 16.0 | 27.9 | 30.2 |
| [Total Omega 3] mg/g | 622.8 | 638.2 | 633.4 | 652.8 | 640.5 |
| DHA/DPAn-3 | 23.4 | 14.7 | 22.6 | 12.0 | 9.3 |
| DPAn-3/DPAn-6 | 1.0 | 1.6 | 1.3 | 2.5 | 3.7 |
| ARA/DPAn-6 | 1.4 | 2.1 | 1.8 | 2.4 | 4.1 |

### Example 8

In this example, Schizochytrium sp. was cultivated, extracted, and further processed under the same conditions as described in Example 1. A biomass level of 149.1 g/L was achieved at the conclusion of the cultivation period. The following tables depict data collected from either the crude microbial oil (Tables 14 and 15) or the final oil after further processing of the crude microbial oil with standard refining, bleaching, winterization, and deodorization procedures (Tables 16 and 17).

TABLE 14
Fatty Acid Methyl Ester (FAME) Profile of Crude Microbial Oil

| Fatty Acid | % Area |
|---|---|
| 12:0 (lauric acid) | 0.2 |
| 14:0 (myristic acid) | 2.2 |
| 16:0 (palmitic acid) | 22 |
| 18:0 (stearic acid) | 1.5 |
| 20:0 (arachidic acid) | 0.4 |
| 20:3n-3 | 0.5 |
| 20:4 (ARA) | 3.1 |
| 20:5 n-3 (EPA) | 37.2 |
| 22:5 n-6 (DPAn-6) | 1.0 |
| 22:5 n-3 (DPAn-3) | 4.6 |
| 22:6 n-3 (DHA) | 23.0 |

TABLE 15
Additional Analysis of Crude Microbial Oil

| [EPA] | 345 | mg/g |
|---|---|---|
| [DHA] | 212 | mg/g |
| EPA/DHA | 1.6 | |
| [Total Omega 3 Fatty Acids] | 598 | mg/g |
| [12:0 (lauric acid)] | 2 | mg/g |
| [14:0 (myristic acid)] | 20 | mg/g |
| [16:0 (palmitic acid)] | 204 | mg/g |
| [18:0 (stearic acid)] | 14 | mg/g |
| [20:4 (ARA)] | 29 | mg/g |
| [22:5 n-6 (DPAn-6)] | 9 | mg/g |
| [22:5 n-3 (DPAn-3)] | 43 | mg/g |
| % TAG | 93.7 | |
| % 1,2-DAG | 1.5 | |
| % 1,3-DAG | 2.4 | |
| % 1-MAG | <0.1 | |
| % 2-MAG | 0.2 | |
| % DHA in sn-1,3 position | 40.2 | |
| % DHA in sn-2 position | 59.8 | |
| % EPA in sn-1,3 position | 66.2 | |
| % EPA in sn-2 position | 33.8 | |
| % sterol (free) | 0.3 | |
| % sterol ester | 0.2 | |
| [stigmasterol] | 4.35 | mg/g |
| [cholesterol] | 0.87 | mg/g |
| % Free Fatty Acids (FFA) | 1.8 | |
| PV | <0.1 | meq/kg |

| | |
|---|---|
| AV | 2.8 |

TABLE 16
Fatty Acid Methyl Ester (FAME) Profile
of Refined/Final Microbial Oil

| Fatty Acid | % Area |
|---|---|
| 12:0 (lauric acid) | 0.2 |
| 14:0 (myristic acid) | 2.0 |
| 16:0 (palmitic acid) | 20.1 |
| 18:0 (stearic acid) | 1.5 |
| 20:0 (arachidic acid) | 0.4 |
| 20:3n-3 | 0.5 |
| 20:4 (ARA) | 3.0 |
| 20:5 n-3 (EPA) | 36.4 |
| 22:5 n-6 (DPAn-6) | 1.0 |
| 22:5 n-3 (DPAn-3) | 4.5 |
| 22:6 n-3 (DHA) | 21.8 |

TABLE 17
Additional Analysis of Refined/Final Microbial Oil

| [EPA] | 337 | mg/g |
|---|---|---|
| [DHA] | 200 | mg/g |
| EPA/DHA | 1.7 | |
| [Total Omega 3 Fatty Acids] | 579 | mg/g |
| [12:0 (lauric acid)] | 2 | mg/g |
| [14:0 (myristic acid)] | 19 | mg/g |
| [16:0 (palmitic acid)] | 186 | mg/g |
| [18:0 (stearic acid)] | 14 | mg/g |
| [20:4 (ARA)] | 28 | mg/g |
| [22:5 n-6 (DPAn-6)] | 9 | mg/g |
| [22:5 n-3 (DPAn-3)] | 42 | mg/g |
| % TAG | 96.3 | |
| % 1,2-DAG | 1.3 | |
| % 1,3-DAG | 1.7 | |
| % 1-MAG | <0.1 | |
| % 2-MAG | 0.2 | |
| % DHA in sn-1,3 position | 37.4 | |
| % DHA in sn-2 position | 62.6 | |
| % EPA in sn-1,3 position | 65.4 | |
| % EPA in sn-2 position | 34.6 | |
| % sterol (free) | 0.2 | |
| % sterol ester | 0.2 | |
| [stigmasterol] | 2.94 | mg/g |
| [cholesterol] | 0.25 | mg/g |
| [3-MCPD] | 0.067 | mg/kg |
| [glycidol] | <0.02 | mg/kg |
| % Free Fatty Acids (FFA) | <0.1 | |
| PV | 0.5 | meq/kg |

| | |
|---|---|
| AV | 1.0 |

### Example 9

The crude oil produced in Example 8 having 345 mg/g EPA and 212 mg/g DHA was further concentrated to produce a nutritional oil concentrate. In further detail, a quantity of the crude oil was transesterified using a solution of sodium ethoxide dissolved in ethanol and the ethyl ester fraction was separated from the glycerol fraction to produce an ethylated oil. The ethylated oil (EE Feed Material) had a concentration of EPA ethyl esters (EPA-EE) of 342.4 mg/g and DHA ethyl esters (DHA-EE) of 203.7 mg/g.

The ethylated oil was passed twice through a VTA fractional distillation apparatus and then through a short path distillation apparatus to achieve the EPA-EE and DHA-EE fractions presented in Table 18. Specifically, the EE Feed Material was passed through the fractional distillation apparatus to separate lower molecular weight compounds from the EPA-EE- and DHA-EE-containing residue. The residue was then passed through the fractional distillation apparatus to separate EPA-EE (Pass 2 Distillate) and DHA-EE (Pass 2 Residue) fractions. The Pass 2 Residue including the high DHA-EE fraction was then further concentrated via short path distillation to remove high molecular weight compounds (boiler fuel) from the DHA-EE fraction. As can be seen in Table 18, a nutritional oil concentrate having greater than 755 mg/g EPA-EE (Pass 2 Distillate) can be produced from a nutritional oil as described herein.

TABLE 18
EPA-EE and DHA-EE Concentrations in Various
Fractions of Concentration Process

| Process Step | [EPA-EE] mg/g | [DHA-EE] mg/g |
|---|---|---|
| EE Feed Material | 342.4 | 203.7 |
| Pass 1 - VTA Distillate | 1.75 | 0 |
| Pass 1 - VTA Residue | 456.2 | 271.6 |
| Pass 2 - VTA Distillate | 755.1 | 25.5 |
| Pass 2 - VTA Residue | 0 | 638.9 |
| Pass 3 - SP Distillate | 0.26 | 697.6 |
| Pass 3 - SP Residue | 0 | 4.7 |

### Example 10

An EPA-EE concentrate produced as in Example 9 was re-esterified to form a re-esterified triglyceride product. In further detail, re-esterified triglycerides were formed by the conversion of ethyl esters back to triglycerides via enzyme-catalyzed glycerolysis. Specifically, 26.0 g of glycerol was combined with 250.0 g of ethyl ester (EE) concentrate using a catalytic amount of Candida antarctica lipase B to produce a re-esterified triglyceride (rTG) concentrate. The potency and lipid class analysis of both the EE concentrate and the rTG concentrate can be seen in Table 19 below.

TABLE 19
Lipid Class and Potency of Re-Esterified Triglyceride

| | Lipid Class (Area %) | | | | | Potency (mg/g) | |
|---|---|---|---|---|---|---|---|
| Sample | Oligomers | TAG | DAG | MAG | EE/FFA | EPA | DHA |
| EPA-EE | 0.0 | 0.0 | 0.0 | 0.0 | 100 | 722.8 | 22.7 |
| rTG Product | 1.6 | 71.1 | 25.3 | 1.6 | 0.3 | 728.3 | 35.7 |

### Example 11

Additional nutritional lipid concentrates were produced as described in Example 9. Specifically, three nutritional oil concentrates (e.g., from Pass 1, Pass 2, and Pass 3) were produced from a nutritional oil as described herein having 349 mg/g EPA and 229 mg/g DHA. The concentrates were characterized and the measured values for these concentrates are presented below in Table 20.

TABLE 20
Nutritional Oil Concentrates Produced
from a Common Nutritional Oil Input

| | Concentrate 1 | Concentrate 2 | Concentrate 3 |
|---|---|---|---|
| EPA-EE (mg/g) | 756 | 81 | 486 |
| DHA-EE (mg/g) | 56 | 601 | 275 |
| Total Omega-3 | 843 | 812 | 832 |
| (mg/g) | | | |
| Anisidine Value (AV) | 11 | 3 | 8 |
| Peroxide Value (PV) | 1 | 1.6 | 2.2 |

The nutritional oil concentrates presented in Table 20 and two additional high-EPA nutritional oil concentrates were each evaluated to determine a variety of other C20 and C22 fatty acids that might also be present in the various concentrates. These values were compared against 9 different fish oils to see how what C20 and C22 fatty acids are present in those oils. Of note, C20:1n-9 was consistently present in each of the fish oil concentrates, but was consistently missing in the microbial oils. The details are shown in Table 21 below (percentages are area %).

TABLE 21
Example C20 and C22 Fatty Acids in Microbial and Fish Oils

| | % | mg/g | % | % | | |
|---|---|---|---|---|---|---|
| | C20:1n- | C20:1n- | C22:1n- | C22:1n- | % | % |
| Sample | 9 | 9 | 11 | 9 | EPA | DHA |
| Concentrate 1 | 0 | 0 | 0 | 0 | 78.77 | 5.78 |
| Concentrate 2 | 0 | 0 | 0 | 0 | 9.26 | 68.56 |
| Concentrate 3 | 0 | 0 | 0 | 0 | 52.84 | 29.53 |
| Concentrate 4 | 0 | 0 | 0 | 0 | 76.44 | 2.58 |
| Concentrate 5 | 0 | 0 | 0 | 0 | 52.16 | 1.07 |
| Fish Conc. 1 | 3.99 | 41.05 | 0 | 0 | 70.70 | 0.135 |
| Fish Conc. 2 | 4.63 | 48.44 | 0 | 0 | 76.59 | 0.074 |
| Fish Conc. 3 | 3.82 | 37.19 | 0 | 0 | 66.80 | 0.559 |
| Fish Conc. 4 | 3.22 | 34.41 | 0 | 0 | 57.32 | 1.47 |
| Fish Conc. 5 | 4.55 | 45.92 | 0 | 0 | 72.00 | 0.276 |
| Fish Conc. 6 | 4.39 | 43.91 | 0 | 0 | 72.04 | 0.38 |
| Fish Conc. 7 | 3.55 | 36.94 | 0 | 0 | 73.26 | 1.87 |
| Fish Conc. 8 | 3.56 | 33.66 | 0 | 0 | 55.29 | 1.59 |
| Fish Conc. 9 | 5.40 | 37.14 | 0 | 0 | 74.12 | 2.74 |

1. (canceled)

2-92. (canceled)

93. A nutritional oil composition, comprising:
triglycerides in an amount of 70 wt. % or greater based on a total weight of the nutritional oil composition;
EPA and DHA present at a weight ratio of greater than or equal to 1.4 EPA/DHA;
palmitic acid in an amount greater than or equal to 30 mg/g based on the total weight of the nutritional oil composition,
stigmasterol, and
DPA n-3 present at a weight ratio with DHA of at least 1.3 DHA/DPA n-3,
wherein the nutritional oil composition has an anisidine value of less than 20.

94. The nutritional oil composition of claim 93, wherein EPA and DHA are present in the nutritional oil composition in a combined amount of from 400 mg/g to 700 mg/g based on the total weight of the nutritional oil composition.

95. The nutritional oil composition of claim 94, wherein palmitic acid is present in the nutritional oil composition in an amount of from 90 mg/g to 230 mg/g based on the total weight of the nutritional oil composition.

96. The nutritional oil composition of claim 95, wherein EPA and DHA are present in the nutritional oil at a weight ratio from 1.4 to 2.4 EPA/DHA.

97. The nutritional oil composition of claim 96, wherein EPA and DHA are present in the nutritional oil at a weight ratio from 1.6 to 2.0 EPA/DHA, or from 1.8 to 2.2 EPA/DHA, or from 2.0 to 2.4 EPA/DHA.

98. The nutritional oil composition of claim 96 wherein EPA and DHA are present in the nutritional oil composition in a combined amount of from 450 mg/g to 650 mg/g based on the total weight of the nutritional oil composition.

99. The nutritional oil composition of claim 96, wherein greater than or equal to 50 wt % of the combination of EPA and DHA of the nutritional oil is produced from a single microorganism source, and wherein the microorganism source is a Thraustochytrid.

100. The nutritional oil composition of claim 96, wherein DPAn-3 is present in the nutritional oil composition in an amount not greater than 100 mg/g based on the total weight of the nutritional oil composition.

101. The nutritional oil composition of claim 96, wherein DHA and DPAn-3 are present in the nutritional oil composition at a weight ratio of 1.5-2.5 DHA/DPA n-3, and wherein the triglycerides are present in an amount of 70-95 wt. % based on a total weight of the nutritional oil composition.

102. The nutritional oil composition of claim 101, wherein the nutritional oil composition comprises cholesterol in an amount not greater than 10 mg/g based on the total weight of the nutritional oil.

103. A nutritional oil composition, comprising:
triglycerides in an amount of 70 wt. % or greater based on a total weight of the nutritional oil composition;
EPA in an amount greater than or equal to 200 mg/g based on a total weight of the nutritional oil composition;
DHA in an amount greater than or equal to 125 mg/g based on the total weight of the nutritional oil composition;
palmitic acid in an amount greater than or equal to 30 mg/g based on the total weight of the nutritional oil composition;
stigmasterol; and
an antioxidant,
wherein EPA and DHA are present at a weight ratio of at least 1.4 EPA/DHA, and
wherein the nutritional oil composition has an anisidine value of less than 20.

104. The nutritional oil composition of claim 103, wherein EPA is present in the nutritional oil composition in an amount from 200 mg/g to 450 mg/g based on the total weight of the nutritional oil composition.

105. The nutritional oil composition of claim 103, wherein DHA is present in the nutritional oil composition in an amount not greater than 300 mg/g.

106. The nutritional oil composition of claim 103, wherein EPA and DHA are present in the nutritional oil at a weight ratio from 1.6 to 2.4 EPA/DHA.

107. The nutritional oil composition of claim 103, wherein palmitic acid is present in the nutritional oil composition in an amount from 30 mg/g to 230 mg/g based on the total weight of the nutritional oil composition.

108. The nutritional oil composition of claim 106, wherein EPA and DHA are present in the nutritional oil at a weight ratio from 1.6 to 2.0 EPA/DHA, or from 1.8 to 2.2 EPA/DHA, or from 2.0 to 2.4 EPA/DHA.

109. The nutritional oil composition of claim 103, wherein greater than or equal to 50 wt % of the combination of EPA and DHA of the nutritional oil is produced from a single microorganism source, and wherein the microorganism source is a species from a genus selected from the group consisting of Schizochytrium, Thraustochytrium, Aurantiochytrium, and Ulkenia.

110. The nutritional oil composition of claim 106, wherein the nutritional oil composition has a peroxide value of less than 5 meq/kg based on the total weight of the nutritional oil composition, wherein the nutritional oil composition further comprises DPA n-3 at a weight ratio with DHA of at least 1.3 DHA/DPA n-3, wherein the triglycerides are present in an amount of 90 wt. % or greater based on a total weight of the nutritional oil composition, and wherein the nutritional oil composition has an anisidine value of less than 15.

111. The nutritional oil composition of claim 103, wherein the nutritional oil composition has a 3-MCPD content of less than 1 mg/kg based on the total weight of the nutritional oil composition.

112. The nutritional oil composition of claim 103, wherein the nutritional oil composition has a glycidol content of less than 1 mg/kg based on the total weight of the nutritional oil.

113. A nutritional oil composition, comprising:
70 wt. % or greater of triglycerides based on a total weight of the nutritional oil composition;
EPA in an amount greater than or equal to 200 mg/g based on the total weight of the nutritional oil composition;
DHA present at a weight ratio with EPA of at least 1.4 EPA/DHA;
stigmasterol; and
DPA n-6,
wherein the nutritional oil composition has a peroxide value of less than or equal to 5 meq/kg based on the total weight of the nutritional oil composition, and wherein the nutritional oil composition has an anisidine value of less than 20.

114. The nutritional oil composition of claim 113, wherein the nutritional oil composition comprises greater than or equal to 80% triglycerides based on the total weight of the nutritional oil composition.

115. The nutritional oil composition of claim 113, wherein EPA is present in the nutritional oil composition in an amount from 250 mg/g to 450 mg/g based on the total weight of the nutritional oil composition.

116. The nutritional oil composition of claim 113, wherein EPA and DHA are present in the nutritional oil at a weight ratio from 1.6 to 2.0 EPA/DHA, or from 1.8 to 2.2 EPA/DHA, or from 2.0 to 2.4 EPA/DHA.

117. The nutritional oil composition of claim 113, further comprising DPA n-3 at a weight ratio with DHA of at least 1.3 DHA/DPA n-3, and wherein the triglycerides are present in an amount of 90 wt. % or greater based on a total weight of the nutritional oil composition.

118. The nutritional oil composition of claim 113, wherein the nutritional oil comprises at least 50 wt % of a microbial oil extracted from a microorganism biomass.

119. The nutritional oil composition of claim 118, wherein greater than or equal to 50 wt % of the combination of EPA and DHA of the nutritional oil is produced from the single microorganism source.

120. The nutritional oil composition of claim 119, wherein the microorganism source is a Thraustochytrid.

121. The nutritional oil composition of claim 120, wherein the microorganism source is a species from a genus selected from the group consisting of Schizochytrium, Thraustochytrium, Aurantiochytrium, and Ulkenia.

122. The nutritional oil composition of claim 113, wherein the nutritional oil comprises a microbial oil blended with a second oil comprising a second microbial oil, a plant oil, or a combination thereof.
